SEQUENCE LISTING (14 sequences)

SEQ ID NO: 1 - DNA, 2299 bp, Homo sapiens
cctaccggtccatagtgtcagagtggtgaacccctgcagccagcaggcctcctgaaaaaaaagtccatgggtgacagaagattcattgacttccaattccaagattcaaattcaagcctcagacccaggttgggcaatgctactgccaataatacttgcattgttgatgattccttcaagtataatctcaatggtgctgtctacagtgttgtattcatcttgggtctgataaccaacagtgtctctctgtttgtcttctgtttccgcatgaaaatgagaagtgagactgctatttttatcaccaatctagctgtctctgatttgctttttgtctgtacactaccttttaaaatattttacaacttcaaccgccactggccttttggtgacaccctctgcaagatctctggaactgcattccttaccaacatctatgggagcatgctctttctcacctgtattagtgtggatcgtttcctggccattgtctatccttttcgatctcgtactattaggactaggaggaattctgccattgtgtgtgctggtgtctggatcctagtcctcagtggcggtatttcagcctctttgttttccaccactaatgtcaacaatgcaaccaccacctgctttgaaggcttctccaaacgtgtctggaagacttatttatccaagatcacaatatttattgaagttgttgggtttatcattcctctaatattgaatgtctcttgctcttctgtggtgctgagaactcttcgcaagcctgctactctgtctcaaattgggaccaataagaaaaaagtactgaaaatgatcacagtacatatggcagtctttgtggtatgctttgtaccctacaactctgtcctcttcttgtatgccctggtgcgctcccaagctattactaattgctttttggaaagatttgcaaagatcatgtacccaatcaccttgtgccttgcaactctgaactgttgttttgaccctttcatctattacttcacccttgaatcctttcagaagtccttctacatcaatgcccacatcagaatggagtccctgtttaagactgaaacacctttgaccacaaagccttcccttccagctattcaagaggaagtgagtgatcaaacaacaaataatggtggtgaattaatgctagaatccaccttttaggtatgagaaatgtgttcaggtccagatatggtttctcctataatttttcctatgctataaactaaagatttgaagctaatgatactgagaataatgcaccaaatccagtcagatacatttgtttgaaggtatactgtagagtttttattgctgttttgttcagtaattataggtcaaatctaattacaacaaccaagatggattgccaaactcttctgcttggttggaatttcattgtatcgcattatccaggtggctagtggcatttgataatatagagatgactttgaaactttcaaaaaggtatttctattccaatgatatttggtaattaggttgggcctataaatatagaacaaattcagggatttttaaaaaattgtgttactactgatatatgctagttttattttatttttttggactgtcattgagtttattttagcacaagaatatttttagcctaacattattaataagaaatgtgtcaaatttttaacattggtaaaatatgttatgtgcattttgaaaacagaaaacaaattgcgttggcatgtacgtgggtgggaagaaaaagaaaattaacaggatttacacaattataatcaccagcagtgtgagtttaaaaaacttcgttgtttttacaccaaattaaaattttcatgtcaaacttcaaagccagaaagctgctaaatacgtgtctggcaggtaaaagctggaaaattacttaaaacaggaaagtgtcaataaaaaaacttgagcaacaccaacatattttttcttaaaatgtcacgttatcttcattttgggaaactaggttctataaaatatttatcctccctgttatactttggagcacagcacagccagaaaggggctgcatttgtgcccaggtcaggagcaaattgaaaaaaaaaataaagtaatactaaaaaatcaaactataaacccaaaacatttattaaaacctgaattaatcctttttggagggaggagtagagatatataacctgaaaatacttattctttcttatcgaattttggagcctaatatagccaggagctgctgaatttgtgcccctggattggaaccaaataaaaaaaaaaaaaaaaaattcct

SEQ ID NO: 2 - protein, 369 aa, Homo sapiens
MGDRRFIDFQFQDSNSSLRPRLGNATANNTCIVDDSFKYNLNGVYSVVFILGLITNSVSLFVFCFRMKMRSETAIFITNLAVSDLLFVCTLPFKIFYNFNRHWPFGDTLCKISGTAFLTNIYGSMLFLTCISVDRFLAIVYPFRSRTIRTRRNSAIVCAGVWILVLSGGISASLFSTTNVNNATTTCFEGFSKRVWKTYLSKITIFIEVVGFIIPLILNVSCSSVVLRTLRKPATLSQIGTNKKKVLKMITVHMAVFVVCFVPYNSVLFLYALVRSQAITNCFLERFAKIMYPITLCLATLNCCFDPFIYYFTLESFQKSFYINAHIRMESLFKTETPLTTKPSLPAIQEEVSDQTTNNGGELMLESTF

SEQ ID NO: 3 - DNA, 2741 bp, Mus musculus
tagactttgggccttttcttgtgtcctgtttgttaaaggcatgcgggctccagcattaaagagggctagtccttaacaaagggaaagcgataaatgtaaataagctcacattttcagaatgagcggtttgcagtaaggagctgcggcagcccagagtctgctctttttgggctgggctaacctttccctgttttttgttttttgttttgttttgtttttgttttttatggataaaaatatgcgcttccgaagtgcgagttgccagtttacacgtttattagctaactatctacaggcatgagcacattctctcatctagcacactctttcttgggcactcaattgaggaactctctgatcgtctgcctccagaaaattcattgattatccaagtctcagataaatctggtgccagagtttggtttgaactaactaatgaagaaagcattctctactggtcctcagtctcaagagtggtgaacccctgcacctagcaggctctctgggaaaaaaaaatccatgggtgacagaagatttattgacttccaattccaagatttaaattcaagtctcagacccaggttgggaaatgcaactgccaataatacttgcattgttgatgattccttcaagtataatttgaatggtgctgtctatagtgttgtattcatcctgggtctaataaccagcagtgcctccctgtttgtcttctgcttccgcatgaaaatgagaagtgagacggctattttcatcaccaacctggccctctctgatttgctttttgtttgtaccctacctttcaaaatattttacaactttaatcgccactggccttttggtgacaccctctgtaagatctcagggactgcgttcctcaccaacatctatgggagcatgctcttcctcacctgcatcagtgtggatcgtttcctagccattgtctatcccttccgatcgcgtaccatcaggaccaggaggaattccgccattgtgtgcgctggagtctggatcctagtcctcagtggtggtatttcagcttctttgttctccaccactaatgtcaacaatgcgaccaccacttgctttgaaggcttctccaaacgtgtctggaagacatacctgtccaagatcactatattcattgaagttgttggattcatcattcctctgatattgaatgtttcttgttcttctgtggtgcttagaaccctccgcaagcctgcaacattgtctcagattgggaccaataaaaaaaaagtgttgaagatgatcacagtgcatatggcagtgtttgtggtatgctttgtaccatacaactccgttctctttttatatgccttggtacgctcccaagccattactaattgcttattggaaaggtttgcaaagatcatgtacccaattaccttgtgccttgcaactctgaattgttgctttgatccttttatctattacttcactcttgaatcctttcagaagtccttttatatcaatacacatataaggatggagtcgctgtttaagactgagacacctctgacccccaaaccttcccttccagctatccaagaggaagttagtgatcaaacaacaaataatggtggtgaattaatgctggaatccaccttctaggtaccagaattgtctttcaggttcagctacagtgtctcttatgatttttttcctatgctataaataggagaaacaaattgaagctaatgatactgagaatagagtaatgtaccaaatgcagtcagatacatttgtttgaacactattgtacatattctgttttgttcagtaattataggtcaagtctaattacaacaaccaaaacagatcagcctcttctgttgagttgacttttcattacctaaatgaccagtggtcttgacttttagtgatgtgagggttatttttaaacttaaaaaaaaaggcattccagtaattttggtaattgggttgggcctataaatatagaacaaattcagggattatttaaaaacatctgtgttactactgatatatgctagtatttttttccttttttgaattaatattgaatttattttaaaaaaagaactatttttacctaatcttaataagacatactgagaaagagaaatgtgttgaattttaaaatattggcaaattttacctagattttaaaaacctaaatgaagtgtttgaatgaatatgggtgggaaatttggaatttagacaacatttacgcatttataataaccacaattagtgtcagcttttaaaactttctttttaaaataattctagaattttcatatgaaattgttaatcctgaaaggtgctacttatgtgcctggcaggtataaaatggaaaactcataaaattaacagtgtcaatttaaaaaaaaaaaaacttcaagcaacactatattatttcttaagattttcatttatcctttatgggggtggggattggcttgtagaaaatatttattcttcatgttaaatgttggggacacattacagccagagagctacagtatttgtgcccaggtcaggagtaaattgaaaaagtaagtgaatagaatagtagcagcaagatatcttaaagcttatattagtagtttttaaggtggtggttagatagctgtaattttgaaatccatactctcttctgtacattttggagcacattttacccaaggccgctgctgaatttgtgctcaggtcgggagcatattgaaaaagatgtgtac

SEQ ID NO: 4 - protein, 370 aa, Mus musculus
MGDRRFIDFQFQDLNSSLRPRLGNATANNTCIVDDSFKYNLNGAVYSVVFILGLITSSASLFVFCFRMKMRSETAIFITNLALSDLLFVCTLPFKIFYNFNRHWPFGDTLCKISGTAFLTNIYGSMLFLTCISVDRFLAIVYPFRSRTIRTRRNSAIVCAGVWILVLSGGISASLFSTTNVNNATTTCFEGFSKRVWKTYLSKITIFIEVVGFIIPLILNVSCSSVVLRTLRKPATLSQIGTNKKKVLKMITVHMAVFVVCFVPYNSVLFLYALVRSQAITNCLLERFAKIMYPITLCLATLNCCFDPFIYYFTLESFQKSFYINTHIRMESLFKTETPLTPKPSLPAIQEEVSDQTTNNGGELMLESTF

SEQ ID NO: 5 - DNA, 25 bp, Homo sapiens
atcttgggtctgataaccaacagtg

SEQ ID NO: 6 - DNA, 24 bp, Homo sapiens
gacgatgagacagagtttaaccct

SEQ ID NO: 7 - DNA, 24 bp, Mus musculus
catgaaaatgagaagtgagacggc

SEQ ID NO: 8 - DNA, 25 bp, Mus musculus
caaacaccatacgaaacatggtatg

SEQ ID NO: 9 - DNA, 20 bp, Mus musculus
ctcagtatcattagcttcaa

SEQ ID NO: 10 - DNA, 20 bp, Mus musculus
ccttccctgaaggttcctcc

SEQ ID NO: 11 - DNA, 57 bp, Homo sapiens
ggggtaccaccatggattacaaggatgacgacgataagggtgacagaagattcattg

SEQ ID NO: 12 - DNA, 35 bp, Homo sapiens
cgatcttaggtggaaaatccgccggcgtaagaata

SEQ ID NO: 13 - DNA, 37 bp, Homo sapiens
acttaagcttggtaccaccatgggtgacagaagattc

SEQ ID NO: 14 - DNA, 38 bp, Homo sapiens
gatcttaggtggaaaatcgccggcgagctcagatctcc

(19) United States
(12) Patent Application Publication
(10) Pub. No.: US 2006/0275285 A1
(43) Pub. Date: Dec. 7, 2006

(54) Methods of inhibiting a GPCR

(75) Inventors: Wei Gu, Simi Valley, CA (US); Melissa Lay Graham, Newbury Park, CA (US); Murielle Veniant-Ellison, Thousand Oaks, CA (US)

Correspondence Address: AMGEN INC., 1120 VETERANS BOULEVARD, SOUTH SAN FRANCISCO, CA, 94080, US

(73) Assignee: Amgen Inc., Thousand Oaks, CA (US)

(21) Appl. No.: 11/414,667

(22) Filed: Apr. 27, 2006

Related U.S. Application Data
(60) Provisional application No. 60/676,526, filed on Apr. 28, 2005.

(51) Int. Cl. A61K 39/395; G01N 33/53

(52) U.S. Cl. 424/133.1; 435/7.1

(57) ABSTRACT

The invention provides methods of identifying modulators, for example, inhibitors, of a G-protein coupled receptor. The modulators can be used for the treatment or prevention of metabolic disorders such as dyslipidemia, metabolic syndrome and obesity. The invention also provides methods of treating or preventing metabolic disorders by administering modulators of G-protein coupled receptor function.

### CROSS-REFERENCE TO RELATED APPLICATIONS

This application claims the benefit of U.S. Provisional Application No. 60/676,526 filed Apr. 28, 2005, which is incorporated herein by reference in its entirety.

### BACKGROUND OF THE INVENTION

G-protein coupled receptors (GPCRs) are cell surface receptors that transduce extracellular signals to downstream effectors, e.g., intracellular signaling proteins, enzymes, or channels. Changes in the activity of these effectors then mediate subsequent cellular events. The interaction between the receptor and the downstream effector is mediated by a G-protein, a heterotrimeric protein that binds GTP. Examples of mammalian G proteins include Gi, Go, Gq, Gs, and Gt. GPCRs typically have seven transmembrane regions, along with an extracellular domain and a cytoplasmic tail at the C-terminus. These receptors form a large superfamily of related receptor molecules that play a key role in many signaling processes, such as sensory and hormonal signal transduction (for a review, see, e.g., Morris and Malbon, Physiol. Reviews 79: 1373-1430; 1999).

Characterization of the human genome has revealed more than 365 genes that encode GPCRs. GPCRs are referred to as “orphan GPCRs” when their endogenous ligands are not known. Frequently, discovery of the endogenous ligand for a GPCR is useful in helping to characterize the function of the GPCR and in the discovery of therapeutics that modulate that function. Certain lipids (e.g., sphingosine 1-phosphate (S1P), lysophosphatidic acid (LPA), free fatty acids and eicosatetraenoic acid) have been identified as endogenous ligands for some members of the GPCR superfamily, including GPR3, GPR6, GPR12, GPR23 and GPR63 (see, e.g., Im, J. Lipid Res. 45:410-18 (2004)).

The further identification of the role of GPCRs in pathologic processes is important in the development of diagnostics as well as the identification of therapeutic agents.

### BRIEF SUMMARY OF THE INVENTION

This invention is based, in part, on the discovery that the GPCR known in the literature as GPR23 is associated with metabolic disorders. Inhibition of GPR23 signaling with a modulator (e.g., a small molecule antagonist or a neutralizing antibody) may be therapeutically beneficial in the treatment of, for example, dyslipidemia, metabolic syndrome and obesity.

The invention provides methods of identifying an inhibitor of GPR23. In one embodiment, the method comprises: contacting a candidate inhibitor with a polypeptide comprising at least 15 contiguous amino acids of SEQ ID NO:2 or SEQ ID NO:4; determining the functional effect of the compound; and selecting a compound that inhibits GPR23. In other embodiments, the polypeptide comprises sometimes at least 25, 50, 100, 150, 200, 250, 300 or 350, contiguous amino acids. In one embodiment, the polypeptide comprises SEQ ID NO:2 or SEQ ID NO:4. Often, the step of determining the functional effect comprises measuring changes in intracellular calcium.

In other embodiments, the methods comprise a step of administering the compound to an animal; determining the effect of the compound on the onset of symptoms of a metabolic disease; and selecting a compound that delays the onset or reduces the severity of the metabolic disease. The metabolic disease is typically dyslipidemia, metabolic syndrome, or obesity. By way of example, the compound can be an antibody (e.g., a neutralizing antibody) or a small molecule (e.g., an inhibitor).

In another aspect, the invention provides a method of treating a metabolic disease or condition, the method comprising administering a GPR23 inhibitor identified using the screening methods described herein. As discussed further below, the present invention contemplates treating or preventing any metabolic disease or condition. The disease can be, e.g., dyslipidemia, metabolic syndrome or obesity.

One embodiment involves a method of identifying a compound to treat or prevent a metabolic disorder, the method comprising:
a) contacting at least one candidate compound with a polypeptide, wherein the polypeptide:
   (i) comprises at least 200 contiguous amino acids of SEQ ID NO:2 or SEQ ID NO:4,
   (ii) has at least 90% identity to a polypeptide of SEQ ID NO:2 or SEQ ID NO:4,
   (iii) comprises SEQ ID NO 2 or SEQ ID NO:4,
   (iv) consists of SEQ ID NO:2 or SEQ ID NO:4; or
   (v) comprises GPR23;
b) determining the functional effect of the candidate compound on the activity of the polypeptide; and
c) selecting a compound from the at least one candidate compound that inhibits the polypeptide.

In some embodiments, the method further comprises the steps of:
d) administering a compound identified in step c) to an animal;
e) determining the effect of the compound on the onset or symptoms of a metabolic disorder; and
f) selecting a compound that delays the onset or reduces the severity of the symptoms of the metabolic disorder.

In some aspects of the invention, the metabolic disorder is selected from the group consisting of dyslipidemia, metabolic syndrome, obesity and obesity-related disorders.

In certain embodiments involving the methods set forth above, the functional effect comprises measuring a change in intracellular calcium or cAMP. In other embodiments, the functional effect comprises performing a binding assay or an inverse agonist assay.

In certain embodiments, the candidate compound is an antibody, whereas it is a small molecule in still other embodiments.

The present invention also contemplates a method of treating a metabolic disorder (e.g., dyslipidemia, metabolic syndrome, obesity and obesity-related disorders) comprising administering a compound, including, but not limited to, an antibody or a small molecule, identified by the method set forth above.

In other aspects, the present invention involves a method of treating a metabolic disorder comprising administering a therapeutically effective amount of a compound that modulates GPR23. The compound, which is an inhibitor in particular embodiments, is often an antibody or a small molecule.

### BRIEF DESCRIPTION OF THE DRAWINGS

FIG. 1 shows that antisense treated animals reduced GPR23 mRNA levels ˜70% relative to control oligo treated animals in white adipose tissue.

FIGS. 2A-C show that antisense oligo treated animals at the 50 mg/kg dose showed a statistically significant decrease in fat mass (FIG. 2A), a statistically significant increase in lean mass (FIG. 2B), and weighed less than control animals (FIG. 2C).

FIG. 3 depicts serum cholesterol levels, as a percentage of change from baseline levels at week 7, in each of the groups.

FIG. 4 indicates that fatty acid synthase mRNA levels were significantly reduced in a dose-dependent manner in white adipose tissue in the antisense oligo treated groups.

### DETAILED DESCRIPTION OF THE INVENTION

GPR23, also referred to as P2Y9 and LPA₄, is a GPCR identified as a receptor for lysophosphatidic acid (LPA). GPR23 is structurally distinct from the other GPCRs that have been identified as receptors for LPA, namely LPA₁, LPA₂, and LPA₃ (EDG-2, EDG-4 and EDG-7, respectively). GPR23 is coupled to Gq and Gs pathways, in contrast to the other LPA receptors (which are coupled to Gi and Go pathways). GPR23 sequence identity between species is high, with the human receptor having more than 96% identity with the murine receptor.

The scientific literature has reported that GPR23 is predominantly expressed in the ovaries, but expression in other organs and tissues has also been observed (see, e.g., Anliker et al., J. Biol. Chem. 279(20):20555-558 (2004) and Noguchi et al., J. Biol. Chem. 278(28):25600-606 (2003)). Patent documents have connected GPR23 to a number of diseases and disorders, including infections (e.g., viral infections), cancer, inflammatory disorders, cardiovascular disorders (e.g., heart failure and hypertension), urological disorders (e.g., urinary retention), and neurological disorders (e.g., anxiety and schizophrenia) (see, e.g., EP 853126; WO 04/106936 and WO 02/068591). However, prior to the present invention, GPR23 has not been shown to be associated with metabolic disorders.

Definitions

“GPCR,” “TGR”, or “GPR23” all refer to a G-protein coupled receptor, the genes for most of which have been mapped to particular chromosomes and which are expressed in particular cell types. These GPCRs have seven transmembrane regions and have “G-protein coupled receptor activity,” e.g., they bind to G-proteins in response to extracellular stimuli and promote production of second messengers such as diacylglycerol (DAG), IP₃, cAMP, and Ca²⁺ via stimulation of downstream effectors such as phospholipase C and adenylate cyclase (for a description of the structure and function of GPCRs, see, e.g., Fong, supra, and Baldwin, supra).

Topologically, GPCRs have an N-terminal “extracellular domain,” a “transmembrane domain” comprising seven transmembrane regions and corresponding cytoplasmic and extracellular loops, and a C-terminal “cytoplasmic domain” (see, e.g., Buck & Axel, Cell 65:175-187 (1991)). These domains can be structurally identified using methods known to those of skill in the art, such as sequence analysis programs that identify hydrophobic and hydrophilic domains (see, e.g., Kyte & Doolittle, J. Mol. Biol. 157:105-132 (1982)). Such domains are useful for making chimeric proteins and for in vitro assays of the invention.

The terms “GPCR” or “GPR23” therefore refer to nucleic acid and polypeptide polymorphic variants, alleles, mutants, and interspecies homologs and GPCR domains thereof that: (1) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a window of at least about 25, 50, 100, 200, 500, 1000, or more amino acids, to a sequence of SEQ ID NO:2 or SEQ ID NO:4; (2) bind to antibodies raised against an immunogen comprising an amino acid sequence of SEQ ID NO:2 or SEQ ID NO:4, and conservatively modified variants thereof, (3) have at least 15 contiguous amino acids, more often, at least 25, 50, 100, 150, 200, 250, 300 or 350 contiguous amino acids, of SEQ ID NO:2 or SEQ ID NO:4; (4) specifically hybridize (with a size of at least 100, preferably at least 500 or 1000 nucleotides) under stringent hybridization conditions to a sequence of SEQ ID NO:1 or SEQ ID NO:3, and conservatively modified variants thereof, (5) have a nucleic acid sequence that has greater than about 95%, preferably greater than about 96%, 97%, 98%, 99%, or higher nucleotide sequence identity, preferably over a region of at least about 50, 100, 200, 500, 1000, or more nucleotides, to SEQ ID NO:1 or SEQ ID NO:3; or (6) are amplified by primers that specifically hybridize under stringent conditions to SEQ ID NO:1 or SEQ ID NO:3. This term also refers to a domain of a GPCR, as described above, or a fusion protein comprising a domain of a GPCR linked to a heterologous protein. A GPR23 polynucleotide or polypeptide sequence of the invention is typically from a mammal including, but not limited to, human, mouse, rat, hamster, cow, pig, horse, sheep, or any mammal. A “GPR23 polynucleotide” and a “GPR23 polypeptide,” are both either naturally occurring or recombinant.

A “full length” GPR23 protein or nucleic acid refers to a polypeptide or polynucleotide sequence, or a variant thereof, that contains all of the elements normally contained in one or more naturally occurring, wild type GPR23 polynucleotide or polypeptide sequences. It will be recognized, however, that derivatives, homologs, and fragments of a GPR23 can be readily used in the present invention.

In some embodiments, the GPCR used in the methods of the invention is a fragment or domain that essentially consists of, at least 15, often at least 25, 50, 100, 150, 200, 250, 300 or 350, contiguous amino acids of SEQ ID NO:2 or SEQ ID NO:4.

“Extracellular domain” refers to the domain of a GPR23 that protrudes from the cellular membrane and often binds to an extracellular ligand. This domain is often useful for in vitro ligand binding assays, both soluble and solid phase.

“Transmembrane domain” comprises seven transmembrane regions plus the corresponding cytoplasmic and extracellular loops. Certain regions of the transmembrane domain can also be involved in ligand binding.

“Cytoplasmic domain” refers to the domain of a GPR23 that protrudes into the cytoplasm after the seventh transmembrane region and continues to the C-terminus of the polypeptide.

“GPR23 activity” refers to the ability of a GPCR to transduce a signal. Such activity can be measured, e.g., in a heterologous cell, by coupling a GPCR (or a chimeric GPCR) to a G-protein and a downstream effector such as PLC or adenylate cyclase, and measuring increases in intracellular calcium (see, e.g., Offermans & Simon, J. Biol. Chem. 270:15175-15180 (1995)). Receptor activity can be effectively measured by recording ligand-induced changes in [Ca²⁺]ᵢ using fluorescent Ca²⁺-indicator dyes and fluorometric imaging. A “natural ligand-induced activity” as used herein, refers to activation of the GPCR by a natural ligand of the GPCR. Activity can be assessed using any number of endpoints to measure the GPCR activity. For example, activity of a GPR23, may be assessed using an assay such as calcium mobilization, e.g., an Aequorin luminescence assay.

The terms “metabolic disease,” “metabolic condition,” “metabolic disorder,” and the like are used interchangeably herein and refer to diabetes, particularly type II diabetes, obesity, obesity-related disorders, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer and edema.

A “host cell” is a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as CHO, HeLa, and the like.

“Biological sample” as used herein is a sample of biological tissue or fluid that contains a GPR23 nucleic acids or polypeptides. Such samples include, but are not limited to, tissue isolated from humans, mice, and rats. Biological samples may also include sections of tissues such as frozen sections taken for histologic purposes. A biological sample is typically obtained from eukaryotic organisms, such as insects, protozoa, birds, fish, reptiles, and preferably mammals such as rats, mice, cows, dogs, guinea pigs, rabbits, and most preferably primates such as chimpanzees or humans. Preferred tissues typically depend on the known expression profile of the GPCR, and include e.g., adipose, leukocytes, neutrophils, monocytes, bone marrow, and spleen.

The phrase “functional effects” in the context of assays for testing compounds that modulate GPR23-mediated signal transduction includes the determination of any parameter that is indirectly or directly under the influence of a GPR23, e.g., a functional, physical, or chemical effect. It includes ligand binding, changes in ion flux, membrane potential, current flow, transcription, G-protein binding, gene amplification, expression in cancer cells, GPCR phosphorylation or dephosphorylation, signal transduction, receptor-ligand interactions, second messenger concentrations (e.g., cAMP, cGMP, IP₃, DAG, or intracellular Ca²⁺), in vitro, in vivo, and ex vivo and also includes other physiologic effects such as increases or decreases of neurotransmitter or hormone release; or increases in the synthesis of particular compounds, e.g., triglycerides.

By “determining the functional effect” is meant assaying for a compound that increases or decreases a parameter that is indirectly or directly under the influence of a GPR23, e.g., functional, physical and chemical effects. Such functional effects can be measured by any means known to those skilled in the art, e.g., changes in spectroscopic characteristics (e.g., fluorescence, absorbance, refractive index), hydrodynamic (e.g., shape), chromatographic, or solubility properties, patch clamping, voltage-sensitive dyes, whole cell currents, radioisotope efflux, inducible markers, transcriptional activation of GPCRs; ligand binding assays; voltage, membrane potential and conductance changes; ion flux assays; changes in intracellular second messengers such as cAMP and inositol triphosphate (IP₃); changes in intracellular calcium levels; neurotransmitter release, and the like.

“Modulators” of a GPR23 refer to inhibitory, activating, or modulating molecules identified using in vitro and in vivo assays for signal transduction, e.g., ligands, agonists, antagonists, and their homologs and mimetics. Such modulating molecules also referred to herein as compounds, include polypeptides, antibodies, amino acids, nucleotides, lipids, carbohydrates, or any organic or inorganic molecule. Inhibitors are compounds that, e.g., bind to, partially or totally block stimulation, decrease, prevent, delay activation, inactivate, desensitize, or down-regulate signal transduction, e.g., antagonists. Activators are compounds that, e.g., bind to, stimulate, increase, open, activate, facilitate, enhance activation, sensitize or up-regulate signal transduction, e.g., agonists. Modulators include compounds that, e.g., alter the interaction of a polypeptide with: extracellular proteins that bind activators or inhibitors; G-proteins; G-protein alpha, beta, and gamma subunits; and kinases. Modulators also include genetically modified versions of a GPR23, e.g., with altered activity, as well as naturally occurring and synthetic ligands, antagonists, agonists, antibodies, small chemical molecules and the like. Such assays for inhibitors and activators include, e.g., expressing a GPR23 in vitro, in cells or cell membranes, applying putative modulator compounds, and then determining the functional effects on signal transduction, as described above.

Samples or assays comprising a GPR23 that are treated with a potential activator, inhibitor, or modulator are compared to control samples without the inhibitor, activator, or modulator to examine the extent of inhibition. Control samples (untreated with inhibitors or other modulating agents) are assigned a relative GPR23 activity value of 100%. Inhibition of a GPR23 is achieved when the GPR23 activity value relative to the control is about 80%, preferably 50%, more preferably 25-0%. Activation of a GPR23 is achieved when the GPR23 activity value relative to the control (untreated with activators) is 110%, more preferably 150%, more preferably 200-500% (i.e., two to five fold higher relative to the control), more preferably 1000-3000% higher.

The terms “isolated”, “purified” or “biologically pure” refer to material that is substantially or essentially free from components, which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated GPR23 nucleic acid is separated from open reading frames that flank the GPR23 gene and encode proteins other than the GPR23. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Biologically active” GPR23 refers to a GPR23 having signal transduction activity and G protein coupled receptor activity, as described above.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide, also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three-dimensional structures within a polypeptide. These structures are commonly known as domains. Domains are portions of a polypeptide that form a compact unit of the polypeptide and are typically 25 to approximately 500 amino acids long. Typical domains are made up of sections of lesser organization such as stretches of β-sheet and α-helices. “Tertiary structure” refers to the complete three-dimensional structure of a polypeptide monomer. “Quaternary structure” refers to the three dimensional structure formed by the noncovalent association of independent tertiary units.

A “label” or a “detectable moiety” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins that can be made detectable, e.g., by incorporating a radiolabel into the peptide, and used to detect antibodies specifically reactive with the peptide).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe.

As used herein a “nucleic acid probe or oligonucleotide” is defined as a nucleic acid capable of binding to a target nucleic acid of complementary sequence through one or more types of chemical bonds, usually through complementary base pairing, usually through hydrogen bond formation. As used herein, a probe may include natural (i.e., A, G, C, or T) or modified bases (7-deazaguanosine, inosine, etc.). In addition, the bases in a probe may be joined by a linkage other than a phosphodiester bond, so long as it does not interfere with hybridization. Thus, for example, probes may be peptide nucleic acids in which the constituent bases are joined by peptide bonds rather than phosphodiester linkages. It will be understood by one of skill in the art that probes may bind target sequences lacking complete complementarity with the probe sequence depending upon the stringency of the hybridization conditions. The probes are preferably directly labeled as with isotopes, chromophores, lumiphores, chromogens, or indirectly labeled such as with biotin to which a streptavidin complex may later bind. By assaying for the presence or absence of the probe, one can detect the presence or absence of the select sequence or subsequence.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “expression vector” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a BLAST or BLAST 2.0 sequence comparison algorithm with default parameters described below, or by manual alignment and visual inspection. Such sequences are then be said to be “substantially identical.” This definition also refers to the complement of a test sequence. Preferably, the identity exists over a region that is at least about 25 amino acids or nucleotides in length, or more preferably over a region that is 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1994-1999).

A preferred example of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as follows: 50% formamide, 5×SSC, and 1% SDS, incubating at 42° C., or, 5×SSC, 1% SDS, incubating at 65° C., with wash in 0.2×SSC, and 0.1% SDS at 65° C. Such washes can be performed for 5, 15, 30, 60, 120, or more minutes. For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include hybridization in a buffer of 40% formamide, 1 M NaCl, 1% SDS at 37° C., and a wash in 1×SSC at 45° C. In some embodiments, the present invention contemplates the use of “highly stringent hybridization conditions” to determine whether nucleic acids will hybridize. Exemplary “highly stringent hybridization conditions” include hybridization to filter-bound nucleic acid in 6×SSC at about 45° C. followed by one or more washes in 0.1×SSC/0.2% SDS at about 68° C. A positive hybridization is at least twice background. Those of ordinary skill will readily recognize that alternative hybridization and wash conditions can be utilized to provide conditions of similar stringency to those set forth above (see, e.g., Ausubel, F. M. et al., eds., 1989, Current Protocols in Molecular Biology, Vol. 1, Green Publishing Associates, Inc. and John Wiley & Sons, Inc., New York, at pp. 6.3.1-6.3.6 and 2.10.3).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term “antibody”, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)). The term antibody also includes bivalent or bispecific molecules, diabodies, triabodies, and tetrabodies. Bivalent and bispecific molecules are described in e.g., in Hollinger et al., Proc. Natl. Acad. Sci. USA 90:6444-6448, 1993; WO9311161; EP404,097; Kostelny et al. J Immunol 148:1547, 1992; Gruber et al., J. Immunol. 152:5368, 1994; Pack and Pluckthun, Biochemistry 31:1579, 1992; Zhu et al., Protein Sci 6:781, 1997; and McCartney, et al., Protein Eng. 8:301, 1995.

For preparation of monoclonal or polyclonal antibodies, any technique known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy (1985)). Techniques for the production of single chain antibodies (see, e.g., U.S. Pat. No. 4,946,778) can be adapted to produce antibodies to polypeptides of this invention. Also, transgenic mice, or other organisms such as other mammals, may be used to express humanized antibodies. Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

An “anti-GPR23” antibody is an antibody or antibody fragment that specifically binds a polypeptide encoded by a GPR23 gene, cDNA, or a subsequence thereof.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to a particular GPR23 can be selected to obtain only those polyclonal antibodies that are specifically immunoreactive with the GPR23, and not with other proteins, except for polymorphic variants, orthologs, and alleles of the GPR23. This selection may be achieved by subtracting out antibodies that cross-react with GPR23 molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background. Antibodies that react only with a particular GPR23 ortholog, e.g., from specific species such as rat, mouse, or human, can also be made as described above, by subtracting out antibodies that bind to the same GPR23 from another species.

The phrase “selectively associates with” refers to the ability of a nucleic acid to “selectively hybridize” with another as defined above, or the ability of an antibody to “selectively (or specifically) bind” to a protein, as defined above.

The term “peptibody” generally refers to molecules comprising at least part of an immunoglobulin Fc domain and at least one peptide. Such peptibodies may be multimers or dimers or fragments thereof, and they may be derivatized.

Peptibodies are known in the art and are described in greater detail in WO 99/25044 and WO 00/24782, which are incorporated herein by reference in their entirety. In general, the description set forth herein regarding the generation and use of antibodies is applicable to peptibodies as well. The peptide used to create the peptibody may be from the amino acid sequence of SEQ ID NOS: 2 and 4.

The terms “treat”, “treating” and “treatment”, as used herein, are meant to include alleviating or abrogating a condition or disease and/or its attendant symptoms. The terms “prevent”, “preventing” and “prevention”, as used herein, refer to a method of delaying or precluding the onset of a condition or disease and/or its attendant symptoms, barring a subject from acquiring a condition or disease or reducing a subject's risk of acquiring a condition or disease.

Isolation of Nucleic Acids Encoding GPR23

A. General Recombinant DNA Methods

This invention relies on routine techniques in the field of recombinant genetics. Basic texts disclosing the general methods of use in this invention include Sambrook & Russell, Molecular Cloning, A Laboratory Manual (3rd Ed, 2001); Kriegler, Gene Transfer and Expression: A Laboratory Manual (1990); and Current Protocols in Molecular Biology (Ausubel et al., eds., 1994-1999). Methods that are used to produce GPCRs for use in the invention may also be employed to produce protein ligands or polypeptides that modulate ligand binding to the receptor, for use in the invention.

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

B. Cloning Methods for the Isolation of Nucleotide Sequences Encoding GPR23

In general, the nucleic acid sequences encoding a GPR23 and related nucleic acid sequence homologs are cloned from cDNA and genomic DNA libraries by hybridization with a probe, or isolated using amplification techniques with oligonucleotide primers, and verified by sequencing. For example, GPR23 sequences are typically isolated from mammalian nucleic acid (genomic or cDNA) libraries by hybridizing with a nucleic acid probe, the sequence of which can be derived from SEQ ID NO:1 or SEQ ID NO:3. Suitable tissues from which GPR23 RNA and cDNA can be isolated include, e.g., immune tissues such as spleen, thymus, peripheral blood leukocytes, and various lymphomas.

Amplification techniques using primers can also be used to amplify and isolate GPR23 nucleic acids from DNA or RNA. Suitable primers can be designed using criteria well known in the art (see, e.g., Dieffenfach & Dveksler, PCR Primer: A Laboratory Manual (1995)). These primers can be used, e.g., to amplify either the full length sequence or a probe of one to several hundred nucleotides, which is then used to screen a mammalian library for a full-length GPR23.

Nucleic acids encoding GPR23 can also be isolated from expression libraries using antibodies as probes. Such polyclonal or monoclonal antibodies can be raised using the sequence of SEQ ID NO:2 or SEQ ID NO:4.

GPR23 polymorphic variants, alleles, and interspecies homologs that are substantially identical to a GPR23 can be isolated using GPR23 nucleic acid probes, and oligonucleotides under stringent hybridization conditions, by screening libraries. Alternatively, expression libraries can be used to clone a GPR23 and polymorphic variants, alleles, and interspecies homologs, by detecting expressed homologs immunologically with antisera or purified antibodies made against a GPR23, which also recognize and selectively bind to the GPR23 homolog. Methods of constructing cDNA and genomic libraries are well known in the art (see, e.g., Sambrook & Russell, supra; and Ausubel et al., supra).

An alternative method of isolating GPR23 nucleic acids and their homologs combines the use of synthetic oligonucleotide primers and amplification of an RNA or DNA template (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify GPR23 nucleic acid sequences directly from mRNA, from cDNA, from genomic libraries or cDNA libraries. Degenerate oligonucleotides can be designed to amplify homologs using the sequences provided herein. Restriction endonuclease sites can be incorporated into the primers. Polymerase chain reaction or other in vitro amplification methods may also be useful, for example, to clone nucleic acid sequences that code for proteins to be expressed, to make nucleic acids to use as probes for detecting the presence of GPR23 mRNA in physiological samples, for nucleic acid sequencing, or for other purposes. Genes amplified by the PCR reaction can be purified from agarose gels and cloned into an appropriate vector.

Gene expression can also be analyzed by techniques known in the art, e.g., reverse transcription and amplification of mRNA, isolation of total RNA or poly A⁺ RNA, northern blotting, dot blotting, in situ hybridization, RNase protection, probing DNA microchip arrays, and the like. In the case where the homologs being identified are linked to a known disease, they can be used with GeneChip™ as a diagnostic tool in detecting the disease in a biological sample, see, e.g., Gunthand et al., AIDS Res. Hum. Retroviruses 14: 869-876 (1998); Kozal et al., Nat. Med. 2:753-759 (1996); Matson et al., Anal. Biochem. 224:110-106 (1995); Lockhart et al., Nat. Biotechnol. 14:1675-1680 (1996); Gingeras et al., Genome Res. 8:435-448 (1998); Hacia et al., Nucleic Acids Res. 26:3865-3866 (1998).

Synthetic oligonucleotides can be used to construct recombinant GPR23 genes for use as probes or for expression of protein. This method is performed using a series of overlapping oligonucleotides usually 40-120 bp in length, representing both the sense and nonsense strands of the gene. These DNA fragments are then annealed, ligated and cloned. Alternatively, amplification techniques can be used with precise primers to amplify a specific subsequence of a GPR23 nucleic acid. The specific subsequence is then ligated into an expression vector.

The nucleic acid encoding a GPR23 is typically cloned into intermediate vectors before transformation into prokaryotic or eukaryotic cells for replication and/or expression. These intermediate vectors are typically prokaryote vectors, e.g., plasmids, or shuttle vectors.

Optionally, nucleic acids encoding chimeric proteins comprising a GPR23 or domains thereof can be made according to standard techniques. For example, a domain such as a ligand binding domain, an extracellular domain, a transmembrane domain (e.g., one comprising seven transmembrane regions and corresponding extracellular and cytosolic loops), the transmembrane domain and a cytoplasmic domain, an active site, a subunit association region, etc., can be covalently linked to a heterologous protein. For example, an extracellular domain can be linked to a heterologous GPR23 transmembrane domain, or a heterologous GPR23 extracellular domain can be linked to a transmembrane domain. Other heterologous proteins of choice include, e.g., green fluorescent protein, luciferase, or β-gal.

C. Expression in Prokaryotes and Eukaryotes

To obtain high level expression of a cloned gene or nucleic acid, such as cDNAs encoding a GPR23, or a protein ligand, one typically subclones a nucleic acid sequence encoding the protein of interest into an expression vector that contains a strong promoter to direct transcription, a transcription/translation terminator, and if for a nucleic acid encoding a protein, a ribosome binding site for translational initiation. Suitable bacterial promoters are well known in the art and described, e.g., in Sambrook & Russell and Ausubel et al., supra. Bacterial expression systems for expressing the protein are available in, e.g., E. coli, Bacillus sp., and Salmonella (Palva et al., Gene 22:229-235 (1983); Mosbach et al., Nature 302:543-545 (1983). Kits for such expression systems are commercially available. Eukaryotic expression systems for mammalian cells, yeast, and insect cells are well known in the art and are also commercially available.

In addition to the promoter, the expression vector typically contains a transcription unit or expression cassette that contains all the additional elements required for the expression of the GPCR encoding nucleic acid in host cells. A typical expression cassette thus contains a promoter operably linked to the nucleic acid sequence encoding a GPCR and signals required for efficient polyadenylation of the transcript, ribosome binding sites, and translation termination. The nucleic acid sequence encoding a GPCR may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette should contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic or prokaryotic cells may be used. Standard bacterial expression vectors include plasmids such as pBR322-based plasmids, pSKF, pET23D, and fusion expression systems such as GST and LacZ. Epitope tags can also be added to recombinant proteins to provide convenient methods of isolation, e.g., c-myc.

Expression vectors containing regulatory elements from eukaryotic viruses are typically used in eukaryotic expression vectors, e.g., SV40 vectors, papilloma virus vectors, and vectors derived from Epstein-Barr virus. Other exemplary eukaryotic vectors include pMSG, pAV009/A⁺, pMTO10/A⁺, pMAMneo-5, baculovirus pDSVE, and any other vector allowing expression of proteins under the direction of the SV40 early promoter, SV40 later promoter, metallothionein promoter, murine mammary tumor virus promoter, Rous sarcoma virus promoter, polyhedrin promoter, or other promoters shown effective for expression in eukaryotic cells.

Some expression systems have markers that provide gene amplification such as thymidine kinase, hygromycin B phosphotransferase, and dihydrofolate reductase. Alternatively, high yield expression systems not involving gene amplification are also suitable, such as using a baculovirus vector in insect cells, with a GPCR-encoding sequence under the direction of the polyhedrin promoter or other strong baculovirus promoters.

The elements that are typically included in expression vectors also include a replicon that functions in E. coli, a gene encoding antibiotic resistance to permit selection of bacteria that harbor recombinant plasmids, and unique restriction sites in nonessential regions of the plasmid to allow insertion of eukaryotic sequences. The particular antibiotic resistance gene chosen is not critical, any of the many resistance genes known in the art are suitable. The prokaryotic sequences are optionally chosen such that they do not interfere with the replication of the DNA in eukaryotic cells, if necessary.

Standard transfection methods are used to produce bacterial, mammalian, yeast or insect cell lines that express large quantities of a GPR23 protein, which are then purified using standard techniques (see, e.g., Colley et al., J. Biol. Chem. 264:17619-17622 (1989); Guide to Protein Purification, in Methods in Enzymology, vol. 182 (Deutscher, ed., 1990)). Transformations of eukaryotic and prokaryotic cells are performed according to standard techniques (see, e.g., Morrison, J. Bact. 132:349-351 (1977); Clark-Curtiss & Curtiss, Methods in Enzymology 101:347-362 (Wu et al., eds, 1983)).

Any of the well known procedures for introducing foreign nucleotide sequences into host cells may be used. These include the use of calcium phosphate transfection, polybrene, protoplast fusion, electroporation, liposomes, microinjection, plasma vectors, viral vectors and any of the other well known methods for introducing cloned genomic DNA, cDNA, synthetic DNA or other foreign genetic material into a host cell (see, e.g., Russell & Sambrook, supra). It is only necessary that the particular genetic engineering procedure used be capable of successfully introducing at least one gene into the host cell capable of expressing a GPR23.

After the expression vector is introduced into the cells, the transfected cells are cultured under conditions favoring expression of a GPR23, which is recovered from the culture using standard techniques identified below.

Transgenic animals, including knockout transgenic animals, that include additional copies of a GPR23 and/or altered or mutated GPR23 transgenes can also be generated. A “transgenic animal” refers to any animal (e.g. mouse, rat, pig, bird, or an amphibian), preferably a non-human mammal, in which one or more cells contain heterologous nucleic acid introduced using transgenic techniques well known in the art. The nucleic acid is introduced into the cell, directly or indirectly, by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA.

In other embodiments, transgenic animals are produced in which expression of a GPR23 is silenced. Gene knockout by homologous recombination is a method that is commonly used to generate transgenic animals. Transgenic mice can be derived using methodologies known to those of skill in the art, see, e.g., Hogan et al., Manipulating the Mouse Embryo: A Laboratory Manual, (1988); Teratocarcinomas and Embryonic Stem Cells. A Practical Approach, Robertson, ed., (1987); and Capecchi et al., Science 244:1288 (1989).

Purification of GPR23

Either naturally occurring or recombinant GPR23s can be purified for use in functional assays. Optionally, recombinant GPR23s are purified. Naturally occurring GPR23s are purified, e.g., from any suitable tissue or cell expressing naturally occurring GPR23s. Recombinant GPR23s are purified from any suitable bacterial or eukaryotic expression system, e.g., CHO cells or insect cells.

A GPR23 may be purified to substantial purity by standard techniques, including selective precipitation with such substances as ammonium sulfate; column chromatography, immunopurification methods, and others (see, e.g., Scopes, Protein Purification: Principles and Practice (1982); U.S. Pat. No. 4,673,641; Ausubel et al., supra; and Russell & Sambrook, supra).

A number of procedures can be employed when a recombinant GPR23 is being purified. For example, proteins having established molecular adhesion properties can be reversibly fused to a GPR23. With the appropriate ligand, a GPR23 can be selectively adsorbed to a purification column and then freed from the column in a relatively pure form. The fused protein is then removed by enzymatic activity. Finally, a GPR23 could be purified using immunoaffinity columns.

Recombinant proteins are expressed by transformed bacteria or eukaryotic cells such as CHO cells or insect cells in large amounts, typically after promoter induction; but expression can be constitutive. Promoter induction with IPTG is one example of an inducible promoter system. Cells are grown according to standard procedures in the art. Fresh or frozen cells are used for isolation of protein using techniques known in the art (see, e.g., Russell & Sambrook, supra; and Ausubel et al., supra).

Immunological Detection of GPCRs and Generation of Antibodies

Antibodies can also be used to detect a GPR23 or can be assessed in the methods of the invention for the ability to inhibit a GPR23. A general overview of the applicable technology can be found in Harlow & Lane, Antibodies: A Laboratory Manual (1988) and Harlow & Lane, Using Antibodies (1999). Methods of producing polyclonal and monoclonal antibodies that react specifically with a GPR23 are known to those of skill in the art (see, e.g., Coligan, Current Protocols in Immunology (1991); Harlow & Lane, supra; Goding, Monoclonal Antibodies: Principles and Practice (2d ed. 1986); and Kohler & Milstein, Nature 256:495-497 (1975)). Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)). Such antibodies can be used for therapeutic and diagnostic applications, e.g., in the treatment and/or detection of any of the GPR23 associated metabolic diseases or conditions described herein.

Humanized forms of non-human (e.g., murine) antibodies may also be used in the methods of the invention. Such antibodies are chimeric immunoglobulins, immunoglobulin chains or fragments thereof (such as Fv, Fab, Fab′, F(ab′)₂ or other antigen-binding subsequences of antibodies). Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are not found in the recipient antibody or in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (see, e.g., Jones et al., Nature 321:522-525, 1986; Riechmann et al., Nature 332:323-329, 1988; and Presta, Curr. Op. Struct. Biol. 2:593-596, 1992).

Human antibodies can also be produced using other techniques known in the art, including phage display libraries (see, e.g., Hoogenboom & Winter, J. Mol. Biol. 227:381, 1991; Marks et al., J. Mol. Biol. 222:581, 1991).

A GPR23 or fragment may be used to produce antibodies specifically reactive with the GPR23. For example, a recombinant GPR23 or an antigenic fragment thereof, is isolated as described herein. Recombinant protein is the preferred immunogen for the production of monoclonal or polyclonal antibodies. Alternatively, a synthetic peptide derived from the sequences disclosed herein and conjugated to a carrier protein can be used as an immunogen. Naturally occurring protein may also be used either in pure or impure form. The product is then injected into an animal capable of producing antibodies. Either monoclonal or polyclonal antibodies may be generated, for subsequent use in immunoassays to measure the protein.

Typically, polyclonal antisera with a titer of 10⁴ or greater are selected and tested for their cross reactivity against non-GPR23 proteins or even other related proteins from other organisms, using a competitive binding immunoassay. Specific polyclonal antisera and monoclonal antibodies will usually bind with a K_{d} of at least about 0.1 mM, more usually at least about 1 μM, optionally at least about 0.1 μM or better, and optionally 0.01 μM or better.

Once GPR23-specific antibodies are available, binding interactions with a GPR23 can be detected by a variety of immunoassay methods. For a review of immunological and immunoassay procedures, see Basic and Clinical Immunology (Stites & Terr eds., 7th ed. 1991). Moreover, the immunoassays of the present invention can be performed in any of several configurations, which are reviewed extensively in Enzyme Immunoassay (Maggio, ed., 1980); and Harlow & Lane, supra.

A GPR23 can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case GPR23 or antigenic subsequence thereof).

Immunoassays also often use a labeling agent to specifically bind to and label the complex formed by the antibody and antigen. The labeling agent may itself be one of the moieties comprising the antibody/antigen complex. Thus, the labeling agent may be a labeled GPR23 polypeptide or a labeled anti-GPR23 antibody. Alternatively, the labeling agent may be a third moiety, such as a secondary antibody, that specifically binds to the antibody/GPR23 complex (a secondary antibody is typically specific to antibodies of the species from which the first antibody is derived). Other proteins capable of specifically binding immunoglobulin constant regions, such as protein A or protein G may also be used as the labeling agent. These proteins exhibit a strong non-immunogenic reactivity with immunoglobulin constant regions from a variety of species (see, e.g., Kronval et al., J. Immunol. 111: 1401-1406 (1973); Akerstrom et al., J. Immunol. 135:2589-2542 (1985)). The labeling agent can be modified with a detectable moiety, such as biotin, to which another molecule can specifically bind, such as streptavidin. A variety of detectable moieties are well known to those skilled in the art.

Commonly used assays include noncompetitive assays, e.g., sandwich assays, and competitive assays. In competitive assays, the amount of a GPR23 present in the sample is measured indirectly by measuring the amount of a known, added (exogenous) GPCR displaced (competed away) from an anti-GPCR antibody by the unknown GPCR present in a sample. Commonly used assay formats include immunoblots, which are used to detect and quantify the presence of protein in a sample. Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5:34-41 (1986)).

The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels, enzymes (e.g., horse radish peroxidase, alkaline phosphatase and others commonly used in an ELISA), and colorimetric labels such as colloidal gold or colored glass or plastic beads (e.g., polystyrene, polypropylene, latex, etc.).

The label may be coupled directly or indirectly to the desired component of the assay according to methods well known in the art. As indicated above, a wide variety of labels may be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecule (e.g., streptavidin), which is either inherently detectable or covalently bound to a signal system, such as a detectable enzyme, a fluorescent compound, or a chemiluminescent compound. The ligands and their targets can be used in any suitable combination with antibodies that recognize GPCRs, or secondary antibodies that recognize anti-GPCR.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that may be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product. Finally, simple colorimetric labels may be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

Cross-Reactivity Determinations

Immunoassays in the competitive binding format can also be used for cross-reactivity determinations. For example, a protein at least partially encoded by SEQ NO: 1 can be immobilized to a solid support. Proteins (e.g., GPR23 proteins and homologs) are added to the assay that competes for binding of the antisera to the immobilized antigen. The ability of the added proteins to compete for binding of the antisera to the immobilized protein is compared to the ability of GPCRs encoded by SEQ ID NO:1 or SEQ ID NO:3 to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of the GPR23, to the immunogen protein (i.e., the GPR23 of SEQ ID NO:2 or SEQ ID NO:4). In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of the protein encoded by SEQ ID NO:1 or SEQ ID NO:3 that is required to inhibit 50% of binding, then the second protein is said to specifically bind to the polyclonal antibodies generated to a GPR23 immunogen.

Assays for Modulators of GPR23

A. Assays for GPR23 Activity

The activity of GPR23 polypeptide can be assessed using a variety of in vitro and in vivo assays to determine functional, chemical, and physical effects, e.g., measuring ligand binding, (e.g., radioactive ligand binding), second messengers (e.g., cAMP, cGMP, IP₃, DAG, or Ca²⁺), ion flux, phosphorylation levels, transcription levels, neurotransmitter levels, and the like. Such assays can be used to test for inhibitors of a GPR23. In particular, the assays can be used to test for compounds that inhibit activator-induced GPR23 activity, for example, by modulating the binding of the ligand to the receptor and/or by modulating the ability of the ligand to activate the receptor. Typically in such assays, the test compound is contacted with a GPR23 in the presence of the activator. The activator may be added to the assay before, after, or concurrently with the test compound. The results of the assay, for example, the level of binding, calcium moblilization, etc. are then compared to the results from a control assay that comprises the GPR23 and the activator in the absence of the test compound.

Screening assays of the invention are used to identify modulators that can be used as therapeutic agents, e.g., antibodies and peptibodies to a GPR23 and small molecule antagonists of GPR23 activity. The present invention contemplates the use of any suitable screening assay (e.g., a binding assay or an inverse agonist assay). Suitable GPCR screening assays, including inverse agonist assays, are well known in the art (see, e.g., WO 05/003786; Takeda et al., Life Sci 74(2-3):367-77 (2003); and Teitler et al., Curr Top Med Chem 2(6):529-38 (2002)). In certain preferred embodiments, the present invention contemplates methods of identifying modulators (e.g., inhibitors) of GPR23 and/or methods of using modulators (e.g., inhibitors) of GPR23 to treat or prevent metabolic disorders (including, for example, dyslipidemia, metabolic syndrome, obesity and obesity-related disorders). However, the methods of the present invention are not limited to embodiments involving inhibitors of GRP23; rather, the present invention contemplates the use of modulators associated with any underlying mechanism of action (e.g., antagonists or agonists) provided that the modulators have a beneficial effect on the treatment or prevention of a metabolic disorder(s). Thus, the methods of identifying compounds and the methods of treatment set forth in the specification and the claims can be performed using compounds besides inhibitors (e.g., agonists).

The effects of test compounds upon the function of the GPR23 polypeptides can be measured by examining any of the parameters described above. Any suitable physiological change that affects GPR23 activity can be used to assess the influence of a test compound on GPR23 activity. When the functional consequences are determined using intact cells or animals, one can also measure a variety of effects such as transmitter release, hormone release, transcriptional changes to both known and uncharacterized genetic markers (e.g., northern blots), changes in cell metabolism such as cell growth or pH changes, and changes in intracellular second messengers such as Ca²⁺, IP₃ or cAMP.

For a general review of GPCR signal transduction and methods of assaying signal transduction, see, e.g., Methods in Enzymology, vols. 237 and 238 (1994) and volume 96 (1983); Bourne et al., Nature 10:349:117-27 (1991); Bourne et al., Nature 348:125-32 (1990); Pitcher et al., Annu. Rev. Biochem. 67:653-92 (1998).

The GPR23 for the assay is often selected from a polypeptide having a sequence of SEQ ID NO:2 or SEQ ID NO:4, or conservatively modified variants thereof. Alternatively, the GPR23 will be derived from a eukaryote and include an amino acid subsequence having amino acid sequence identity to SEQ ID NO:2 or SEQ ID NO:4. Generally, the amino acid sequence identity will be at least 70%, optionally at least 80%, optionally at least 90%, optionally at least 95%, optionally at least 97%, optionally at least 98%, or optionally at least 99%. The GPR23 typically comprises at least 15, often at least 25, 50, 100, 150, 200, 250, 300 or 350, contiguous amino acids of SEQ ID NO:2 or SEQ ID NO:4. Optionally, the polypeptide of the assays will comprise or consist of a domain of a GPR23, such as an extracellular domain, transmembrane domain, cytoplasmic domain, ligand binding domain, subunit association domain, active site, and the like. Either a GPR23 or a domain thereof can be covalently linked to a heterologous protein to create a chimeric protein used in the assays described herein.

Modulators of GPR23 activity are tested using GPR23 polypeptides as described above, either recombinant or naturally occurring. The protein can be isolated, expressed in a cell, expressed in a membrane derived from a cell, expressed in tissue or in an animal, either recombinant or naturally occurring. For example, transformed cells or membranes can be used. Modulation can be evaluated using one of the in vitro or in vivo assays described herein. Signal transduction can also be examined in vitro with soluble or solid state reactions, using a chimeric molecule such as an extracellular domain of a receptor covalently linked to a heterologous signal transduction domain, or a heterologous extracellular domain covalently linked to the transmembrane and/or cytoplasmic domain of a receptor. Furthermore, ligand-binding domains of the protein of interest can be used in vitro in soluble or solid state reactions to assay for ligand binding.

Ligand binding to a GPR23, a domain, or chimeric protein can be tested in a number of formats. Binding can be performed in solution, in a bilayer membrane, attached to a solid phase, in a lipid monolayer, or in vesicles. Typically, in an assay of the invention, the binding of a ligand to a GPR23 is measured in the presence of a candidate modulator. Alternatively, the binding of the candidate modulator may be measured in the presence of the ligand. Often, competitive assays that measure the ability of a compound to compete with binding of the ligand to the receptor are used. Binding can be tested by measuring, e.g., changes in spectroscopic characteristics (e.g., fluorescence, absorbance, refractive index), hydrodynamic (e.g., shape) changes, or changes in chromatographic or solubility properties.

Receptor—G-protein interactions can also be used to assay for inhibitors. For example, in the absence of GTP, binding of an activator will lead to the formation of a tight complex of a G protein (all three subunits) with the receptor. This complex can be detected in a variety of ways, as noted above. Inhibitors may be identified by looking at dissociation of the receptor-G protein complex. In the presence of GTP, release of the alpha subunit of the G protein from the other two G protein subunits serves as a criterion of activation.

An inhibited G-protein will, in turn, alter the properties of downstream effectors such as proteins, enzymes, and channels. The classic examples are the activation of cGMP phosphodiesterase by transducin in the visual system, adenylate cyclase by the stimulatory G-protein, phospholipase C by G_{q} and other cognate G proteins, and modulation of diverse channels by Gi and other G proteins. Downstream consequences such as generation of diacyl glycerol and IP₃ by phospholipase C, and, in turn, for calcium mobilization, e.g., by IP₃ (further discussed below), can also be examined. Thus, modulators can be evaluated for the ability to inhibit downstream effects. For example, candidate inhibitors may be assessed for the ability to inhibit calcium mobilization mediated by a GPR23.

Activated GPCRs become substrates for kinases that phosphorylate the C-terminal tail of the receptor (and possibly other sites as well). Thus, activators will promote the transfer of ³²P from gamma-labeled GTP to the receptor, which can be assayed with a scintillation counter. The phosphorylation of the C-terminal tail will promote the binding of arrestin-like proteins and will interfere with the binding of G-proteins. Inhibitors can be identified by the ability to reduce the transfer of ³²P to the receptor. The kinase/arrestin pathway plays a key role in the desensitization of many GPCR receptors.

Inhibitors may therefore also be identified using assays involving α-arrestin recruitment. β-arrestin serves as a regulatory protein that is distributed throughout the cytoplasm in unactivated cells. Ligand binding to an appropriate GPCR is associated with redistribution of β-arrestin from the cytoplasm to the cell surface, where it associates with the GPCR. Thus, receptor activation and the effect of candidate inhibitors on ligand-induced receptor activation, can be assessed by monitoring β-arrestin recruitment to the cell surface. This is frequently performed by transfecting a labeled β-arrestin fusion protein (e.g., β-arrestin -green fluorescent protein (GFP)) into cells and monitoring its distribution using confocal microscopy (see, e.g., Groarke et al., J. Biol. Chem. 274(33):23263-69 (1999)).

Receptor internalization assays may also be used to assess receptor function. Upon ligand binding, the G-protein coupled receptor-ligand complex is internalized from the plasma membrane by a clathrin-coated vesicular endocytic process; internalization motifs on the receptors bind to adaptor protein complexes and mediate the recruitment of the activated receptors into clathrin-coated pits and vesicles. Because only activated receptors are internalized, it is possible to detect ligand-receptor binding by determining the amount of internalized receptor. In one assay format, cells are transiently transfected with radiolabeled receptor and incubated for an appropriate period of time to allow for ligand binding and receptor internalization. Thereafter, surface-bound radioactivity is removed by washing with an acid solution, the cells are solubilized, and the amount of internalized radioactivity is calculated as a percentage of ligand binding. See, e.g., Vrecl et al., Mol. Endocrinol. 12:1818-29 (1988) and Conway et al., J. Cell Physiol. 189(3):341-55 (2001). In addition, receptor internalization approaches have allowed real-time optical measurements of GPCR interactions with other cellular components in living cells (see, e.g., Barak et al., Mol. Pharmacol. 51(2)177-84 (1997)). Inhibitors may be identified by comparing receptor internalization levels in control cells and cells contacted with candidate compounds. For example, candidate inhibitors are assayed by examining their effects on receptor internalization upon binding of a ligand.

Another technology that can be used to evaluate GPCR-protein interactions in living cells involves bioluminescence resonance energy transfer (BRET). A detailed discussion regarding BRET can be found in Kroeger et al., J. Biol. Chem., 276(16):12736-43 (2001).

Receptor-stimulated guanosine 5′-O-(γ-Thio)-Triphosphate ([³⁵S]GTPγS) binding to G-proteins may also be used as an assay for evaluating modulators of GPCRs. [³⁵S]GTPγS is a radiolabeled GTP analog that has a high affinity for all types of G-proteins, is available with a high specific activity and, although unstable in the unbound form, is not hydrolyzed when bound to the G-protein. Thus, it is possible to quantitatively assess ligand-bound receptor by comparing stimulated versus unstimulated [³⁵S]GTPγS binding utilizing, for example, a liquid scintillation counter. Inhibitors of the receptor-ligand interactions would result in decreased [³⁵S]GTPγS binding. Descriptions of [³⁵S]GTPγS binding assays are provided in Traynor and Nahorski, Mol. Pharmacol. 47(4):848-54 (1995) and Bohn et al., Nature 408:720-23 (2000).

The ability of inhibitors to affect ion flux may also be determined. Ion flux may be assessed by determining changes in polarization (i.e., electrical potential) of the cell or membrane expressing a GPCR. One means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., PFlugers. Archiv. 391:85 (1981). Other known assays include: radiolabeled ion flux assays and fluorescence assays using voltage-sensitive dyes (see, e.g., Vestergarrd-Bogind et al., J. Membrane Biol. 88:67-75 (1988); Gonzales & Tsien, Chem. Biol. 4:269-277 (1997); Daniel et al., J. Pharmacol. Meth. 25:185-193 (1991); Holevinsky et al., J. Membrane Biology 137:59-70 (1994)). Generally, the compounds to be tested are present in the range from 1 pM to 100 mM.

Preferred assays for G-protein coupled receptors include cells that are loaded with ion or voltage sensitive dyes to report receptor activity. Assays for determining activity of such receptors can also use known agonists and antagonists for other G-protein coupled receptors as negative or positive controls to assess activity of tested compounds. In assays for identifying antagonists, changes in the level of ions in the cytoplasm or membrane voltage are monitored using an ion sensitive or membrane voltage fluorescent indicator, respectively. Among the ion-sensitive indicators and voltage probes that may be employed are those disclosed in the Molecular Probes 1997 Catalog. For G-protein coupled receptors, promiscuous G-proteins such as Gα15 and Gα16 can be used in the assay of choice (Wilkie et al., Proc. Nat'l Acad. Sci. USA 88:10049-10053 (1991)). Such promiscuous G-proteins allow coupling of a wide range of receptors to signal transduction pathways in heterologous cells.

As noted above, receptor activation by ligand binding typically initiates subsequent intracellular events, e.g., increases in second messengers such as IP₃, which releases intracellular stores of calcium ions. Activation of some G-protein coupled receptors stimulates the formation of inositol triphosphate (IP₃) through phospholipase C-mediated hydrolysis of phosphatidylinositol (Berridge & Irvine, Nature 312:315-21 (1984)). IP₃ in turn stimulates the release of intracellular calcium ion stores. Thus, a change in cytoplasmic calcium ion levels, or a change in second messenger levels such as IP₃, can be used to assess G-protein coupled receptor function. Cells expressing such G-protein coupled receptors may exhibit increased cytoplasmic calcium levels as a result of contribution from both intracellular stores and via activation of ion channels, in which case it may be desirable, although not necessary, to conduct such assays in calcium-free buffer, optionally supplemented with a chelating agent such as EGTA, to distinguish fluorescence response resulting from calcium release from internal stores.

Other assays can involve determining the activity of receptors which, when activated by ligand binding, result in a change in the level of intracellular cyclic nucleotides, e.g., cAMP or cGMP, by activating or inhibiting downstream effectors such as adenylate cyclase. For example, where activation of a GPR23 receptor results in a decrease in cyclic nucleotide levels, it may be preferable to expose the cells to agents that increase intracellular cyclic nucleotide levels, e.g., forskolin, prior to adding a receptor-activating compound to the cells in the assay. Cells for this type of assay can be made by co-transfection of a host cell with DNA encoding a cyclic nucleotide-gated ion channel, GPCR phosphatase and DNA encoding a receptor (e.g., certain glutamate receptors, muscarinic acetylcholine receptors, dopamine receptors, serotonin receptors, and the like), which, when activated, causes a change in cyclic nucleotide levels in the cytoplasm.

In one embodiment, changes in intracellular cAMP or cGMP can be measured using immunoassays. The method described in Offermanns & Simon, J. Biol. Chem. 270:15175-15180 (1995) may be used to determine the level of cAMP. Also, the method described in Felley-Bosco et al., Am. J. Resp. Cell and Mol. Biol. 11:159-164 (1994) may be used to determine the level of cGMP. Further, an assay kit for measuring cAMP and/or cGMP is described in U.S. Pat. No. 4,115,538, herein incorporated by reference.

In another embodiment, phosphatidyl inositol (PI) hydrolysis can be analyzed according to U.S. Pat. No. 5,436,128, herein incorporated by reference. Briefly, the assay involves labeling of cells with ³H-myoinositol for at least 48 hours. The labeled cells are treated with a test compound for one hour. The treated cells are lysed and extracted in chloroform-methanol-water after which the inositol phosphates are separated by ion exchange chromatography and quantified by scintillation counting. Fold inhibition is determined by calculating the ratio of cpm in the presence of antagonist to cpm in the presence of buffer control (which may or may not contain an agonist).

In another embodiment, transcription levels can be measured to assess the effects of a test compound on ligand-induced signal transduction. A host cell containing the protein of interest is contacted with a test compound in the presence of a ligand for a sufficient time to effect any interactions, and then the level of gene expression is measured. The amount of time to effect such interactions may be empirically determined, such as by running a time course and measuring the level of transcription as a function of time. The amount of transcription may be measured by using any method known to those of skill in the art to be suitable. For example, mRNA expression of the protein of interest may be detected using northern blots or their polypeptide products may be identified using immunoassays. Alternatively, transcription-based assays using reporter genes may be used as described in U.S. Pat. No. 5,436,128, herein incorporated by reference. The reporter genes can be, e.g., chloramphenicol acetyltransferase, firefly luciferase, bacterial luciferase, β-galactosidase and alkaline phosphatase. Furthermore, the protein of interest can be used as an indirect reporter via attachment to a second reporter such as green fluorescent protein (see, e.g., Mistili & Spector, Nature Biotechnology 15:961-964 (1997)).

The amount of transcription is then compared to the amount of transcription in either the same cell in the absence of the test compound in a substantially identical cell that is untreated. A substantially identical cell may be derived from the same cells from which the recombinant cell was prepared but which had not been modified by introduction of heterologous DNA. Any difference in the amount of transcription indicates that the test compound has in some manner altered the activity of the protein of interest.

In assays to identify GPR23 inhibitors, samples that are treated with a potential GPR23 inhibitor are compared to control samples to determine the extent of modulation. Control samples (untreated with candidate inhibitors) are assigned a relative activity value of 100. Inhibition of GPR23 is achieved when the activity value relative to the control is about 90%, optionally 50%, optionally 25-0%.

B. Inhibitors

The compounds tested as inhibitors of GPR23 can be any small chemical compound, or a biological entity, e.g., a macromolecule such as a protein, sugar, nucleic acid or lipid. Alternatively, modulators can be genetically altered versions of GPR23. Typically, test compounds will be small chemical molecules and peptides. Essentially any chemical compound can be used as a potential modulator or ligand in the assays of the invention. Most often, compounds can be dissolved in aqueous or organic (especially DMSO-based) solutions. The assays are designed to screen large chemical libraries by automating the assay steps, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909 6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Russell & Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

C. Solid State and Soluble High Throughput Assays

In one embodiment the invention provides soluble assays using molecules such as a domain, e.g., a ligand binding domain, an extracellular domain, a transmembrane domain (e.g., one comprising seven transmembrane regions and cytosolic loops), the transmembrane domain and a cytoplasmic domain, an active site, a subunit association region, etc.; a domain that is covalently linked to a heterologous protein to create a chimeric molecule; a GPR23; or a cell or tissue expressing a GPR23, either naturally occurring or recombinant. In another embodiment, the invention provides solid phase based in vitro assays in a high throughput format, where the domain, chimeric molecule, GPR23, or cell or tissue expressing GPR23 is attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-1500 different compounds. It is possible to assay several different plates per day; assay screens for up to about 6,000-20,000 different compounds is possible using the integrated systems of the invention.

The molecule of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest (e.g., the signal transduction molecule of interest) is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.). Antibodies to molecules with natural binders such as biotin are also widely available and are appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs, for example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly-gly sequences of between about 5 and 200 amino acids. Such flexible linkers are known to persons of skill in the art. For example, poly(ethelyne glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149-2154 (1963) (describing solid phase synthesis of, e.g., peptides); Geysen et al., J. Immun. Meth. 102:259-274 (1987) (describing synthesis of solid phase components on pins); Frank & Doring, Tetrahedron 44:6031-6040 (1988) (describing synthesis of various peptide sequences on cellulose disks); Fodor et al., Science, 251:767-777 (1991); Sheldon et al., Clinical Chemistry 39(4):718-719 (1993); and Kozal et al., Nature Medicine 2(7):753-759 (1996) (all describing arrays of biopolymers fixed to solid substrates). Non-chemical approaches for fixing tag binders to substrates include other common methods, such as heat, cross-linking by UV radiation, and the like.

D. Computer-Based Assays

Yet another assay for compounds that modulate GPR23 activity involves computer assisted drug design, in which a computer system is used to generate a three-dimensional structure of GPR23 based on the structural information encoded by the amino acid sequence. The input amino acid sequence interacts directly and actively with a pre-established algorithm in a computer program to yield secondary, tertiary, and quaternary structural models of the protein. The models of the protein structure are then examined, for example, to identify the regions that have the ability to bind ligands. These regions are then used to identify various compounds that inhibit ligand-receptor binding.

The three-dimensional structural model of the protein is generated by entering protein amino acid sequences of at least 10 amino acid residues or corresponding nucleic acid sequences encoding a GPR23 polypeptide into the computer system. The amino acid sequence of the GPR23 polypeptide typically comprises SEQ ID NO: 2 or SEQ ID NO:4, or conservatively modified variants of SEQ ID NO:2 or SEQ ID NO:4. The amino acid sequence represents the primary sequence or subsequence of the protein, which encodes the structural information of the protein. At least 10 residues of the amino acid sequence (or a nucleotide sequence encoding 10 amino acids) are entered into the computer system from computer keyboards, computer readable substrates that include, but are not limited to, electronic storage media (e.g., magnetic diskettes, tapes, cartridges, and chips), optical media (e.g., CD ROM), information distributed by internet sites, and by RAM. The three-dimensional structural model of the protein is then generated by the interaction of the amino acid sequence and the computer system, using software known to those of skill in the art.

The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model.

The tertiary structure of the protein encoded by the secondary structure is then formed on the basis of the energy terms of the secondary structure. The user at this point can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like.

Once the structure has been generated, potential ligand binding regions are identified by the computer system. Three-dimensional structures for potential ligands are generated by entering amino acid or nucleotide sequences or chemical formulas of compounds, as described above. The three-dimensional structure of the potential ligand is then compared to that of a GPR23 to identify ligands that bind to the GPR23. Binding affinity between the protein and ligands is determined using energy terms to determine which ligands have an enhanced probability of binding to the protein.

Computer systems are also used to screen for mutations, polymorphic variants, alleles and interspecies homologs of GPR23 genes. Such mutations can be associated with disease states or genetic traits. As described above, GeneChip™ and related technology can also be used to screen for mutations, polymorphic variants, alleles and interspecies homologs. Once the variants are identified, diagnostic assays can be used to identify patients having such mutated genes. Identification of the mutated GPR23 genes involves receiving input of a first nucleic acid or amino acid sequence encoding a GPR23, e.g., SEQ ID NO: 1 or SEQ ID NO:3; or SEQ ID NO:2 or SEQ ID NO:4, respectively, and conservatively modified versions thereof. The sequence is entered into the computer system as described above. The first nucleic acid or amino acid sequence is then compared to a second nucleic acid or amino acid sequence that has substantial identity to the first sequence. The second sequence is entered into the computer system in the manner described above. Once the first and second sequences are compared, nucleotide or amino acid differences between the sequences are identified. Such sequences can represent allelic differences in GPR23 genes, and mutations associated with disease states and genetic traits.

E. Expression Assays

Certain screening methods involve screening for a compound that modulates the expression of GPR23. Such methods generally involve conducting cell-based assays in which test compounds are contacted with one or more cells expressing a GPR23 and then detecting a decrease in expression (either transcript or translation product). Such assays are often performed with cells that express the endogenous GPR23.

Expression can be detected in a number of different ways. As described herein, the expression levels of the protein in a cell can be determined by probing the mRNA expressed in a cell with a probe that specifically hybridizes with a GPR23 transcript (or complementary nucleic acid derived therefrom). Alternatively, protein can be detected using immunological methods in which a cell lysate is probed with antibodies that specifically bind to the protein.

Other cell-based assays are reporter assays conducted with cells that do not express the protein. Often, these assays are conducted with a heterologous nucleic acid construct that includes a promoter that is operably linked to a reporter gene that encodes a detectable product. A number of different reporter genes can be utilized. Some reporters are inherently detectable. An example of such a reporter is green fluorescent protein that emits fluorescence that can be detected with a fluorescence detector. Other reporters generate a detectable product. Often such reporters are enzymes. Exemplary enzyme reporters include, but are not limited to, β-glucuronidase, CAT (chloramphenicol acetyl transferase), luciferase, β-galactosidase and alkaline phosphatase.

In these assays, cells harboring the reporter construct are contacted with a test compound. A test compound that inhibits the activity of the promoter, e.g., by binding to it or triggering a cascade that produces a molecule that decreases the promoter-induced expression of the detectable reporter can be detected by comparison to control cells that have not been treated with the inhibitor. Certain other reporter assays are conducted with cells that harbor a heterologous construct that includes a transcriptional control element that activates expression of GPR23 and a reporter operably linked thereto. Here, too, an agent that binds to the transcriptional control element to activate expression of the reporter or that triggers the formation of an agent that binds to the transcriptional control element to activate reporter expression, can be identified by the generation of signal associated with reporter expression.

Kits

A GPR23, e.g., a recombinant GPR23 or a homolog, is a useful tool for diagnosing metabolic disorder or susceptibility to a metabolic disorders-related disease, and for examining signal transduction. GPR23-specific reagents that specifically bind to GPR23 or GPR23 antibodies are used to examine signal transduction regulation.

The present invention also provides for kits for screening for modulators of ligand-GPCR interactions. Such kits can be prepared from readily available materials and reagents. For example, such kits can comprise any one or more of the following materials: a GPCR, typically a recombinant GPCR, reaction tubes, a nicotinic acid reagent, and instructions for testing GPCR activity. Optionally, the kit contains biologically active GPCR. A wide variety of kits and components can be prepared according to the present invention, depending upon the intended user of the kit and the particular needs of the user.

Treatment and Diagnosis of Metabolic Disorders with GPR23 Modulators

A. Metabolic Disorders

Inhibition of GPR23 activity can delay the onset and/or reduce the symptoms of metabolic disorders. Thus, GPR23 inhibitors can be used to treat various metabolic-related disorders. These disorders include diabetes, particularly type II diabetes, obesity, hyperglycemia, glucose intolerance, insulin resistance, hyperinsulinemia, hypercholesterolemia, hypertension, hyperlipoproteinemia, hyperlipidemia, hypertriglylceridemia, dyslipidemia, metabolic syndrome, syndrome X, cardiovascular disease, atherosclerosis, kidney disease, ketoacidosis, thrombotic disorders, nephropathy, diabetic neuropathy, diabetic retinopathy, sexual dysfunction, dermatopathy, dyspepsia, hypoglycemia, cancer and edema. Various other metabolic disorders are described, e.g., in Harrison's Principles of Internal Medicine, 12th Edition, Wilson, et al., eds., McGraw-Hill, Inc.).

“Obesity” is a condition characterized by an excess of body fat. The operational definition of obesity is based on the Body Mass Index (BMI), which is calculated as body weight per height in meter squared (kg/m²). Obesity refers to a condition whereby an otherwise healthy subject has a BMI greater than or equal to 30 kg/m², or a condition whereby a subject with at least one co-morbidity has a BMI greater than or equal to 27 kg/m². An “obese subject” is an otherwise healthy subject with a BMI greater than or equal to 30 kg/m² or a subject with at least one co-morbidity with a BMI greater than or equal 27 kg/m². A “subject at risk of obesity” is an otherwise healthy subject with a BMI of 25 kg/m² to less than 30 kg/m² or a subject with at least one co-morbidity with a BMI of 25 kg/m² to less than 27 kg/m².

The increased risks associated with obesity may occur at a lower BMI in people of Asian descent. In Asian and Asian-Pacific countries, including Japan, “obesity” refers to a condition whereby a subject with at least one obesity-induced or obesity-related co-morbidity that requires weight reduction or that would be improved by weight reduction, has a BMI greater than or equal to 25 kg/m². An “obese subject” in these countries refers to a subject with at least one obesity-induced or obesity-related co-morbidity that requires weight reduction or that would be improved by weight reduction, with a BMI greater than or equal to 25 kg/m². In these countries, a “subject at risk of obesity” is a person with a BMI of greater than 23 kg/m² to less than 25 kg/m².

The term “obesity-related disorders” encompasses disorders that are associated with, caused by, or result from obesity. Examples of obesity-related disorders include overeating and bulimia, diabetes, hypertension, elevated plasma insulin concentrations and insulin resistance, dyslipidemia, hyperlipidemia, breast, prostate, endometrial and colon cancer, heart disease, cardiovascular disorders, abnormal heart rhythms and arrhythmias, myocardial infarction, congestive heart failure, coronary heart disease, angina pectoris, cerebral infarction, cerebral thrombosis, transient ischemic attack, arthritis deformans, sudden death, osteoarthritis, cholelithiasis, gallstones and gallbladder disease, lumbodynia, emmeniopathy, obstructive sleep apnea, stroke, polycystic ovary disease, craniopharyngioma, the Prader-Willi Syndrome, Frohlich's syndrome, GH-deficiency, normal variant short stature, and Turner syndrome. Other examples include pathological conditions showing reduced metabolic activity or a decrease in resting energy expenditure as a percentage of total fat-free mass, such as in children with acute lymphoblastic leukemia. Further examples of obesity-related disorders include metabolic syndrome, also known as syndrome X, insulin resistance syndrome, impaired fasting glucose, impaired glucose tolerance, reproductive hormone abnormalities, sexual and reproductive dysfunction, such as impaired fertility, infertility, hirsutism in females and hypogonadism in males, fetal defects associated with maternal obesity, gastrointestinal motility disorders, such as obesity-related gastro-esophageal reflux, respiratory disorders, such as obesity-hypoventilation syndrome (Pickwickian syndrome), and breathlessness, fatty liver, dermatological disorders, inflammation, such as systemic inflammation of the vasculature, arteriosclerosis, hypercholesterolemia, hyperuricaemia, lower back pain, orthopedic disorders, gout, kidney cancer and increased anesthetic risk, as well as secondary outcomes of obesity such as left ventricular hypertrophy.

The term “metabolic syndrome,” or syndrome X, as used herein, is present if a person has three or more of the following symptoms: abdominal obesity, hyperglyceridemia, low HDL cholesterol, high blood pressure, and high fasting plasma glucose. The criteria for these symptoms are defined in the 3^{rd} Report of the National Cholesterol Education Program Expert Panel in Detection, Evaluation and Treatment of High blood Cholesterol in Adults (Ford, E. S. et al. (2002), JAMA 287(3): 356-359).

The term “diabetes” includes both insulin-dependent diabetes mellitus (IDDM, or Type I diabetes) and non-insulin dependent diabetes mellitus (NIDDM, or Type II diabetes). Type I diabetes results from an absolute deficiency of insulin, the hormone regulating glucose utilization. Type II diabetes often occurs when levels of insulin are normal or even elevated and appears to result from the inability of tissues to respond appropriately to insulin. Most of the Type II diabetics are also obese. The compositions of the present invention can be used for treating both Type I and II diabetes and for treating and/or preventing gestational diabetes mellitus indirectly by preventing obesity. The compositions of the invention can also be useful for treating and preventing diabetes directly. As used herein, the terms “eating disorder”, “feeding disorder”, and the like refer to an emotional and/or behavioral disturbance associated with an excessive decrease in body weight and/or inappropriate efforts to avoid weight gain, e.g., fasting, self-induced vomiting, laxative or diuretic abuse. Depression is commonly associated with eating disorders. Exemplary eating disorders include anorexia nervosa and bulimia.

B. Administration of GPR23 Modulators and Combination Therapy Therewith

Depending on the disease to be treated and the subject's condition, the inhibitors of GPR23 may be administered by oral, parenteral (e.g., intramuscular, intraperitoneal, intravenous, ICV, intracisternal injection or infusion, subcutaneous injection or implant), inhalation, nasal, vaginal, rectal, sublingual, or topical (e.g., transdermal, local) routes of administration and may be formulated, alone or together, in suitable dosage unit formulations containing conventional non-toxic pharmaceutically acceptable carriers, adjuvants and vehicles appropriate for each route of administration. The invention also contemplates administration of the inhibitors in a depot formulation, in which the active ingredient is released over a defined time period.

In the treatment or prevention of metabolic disorders with a modulator (e.g., antagonist) of GPR23 function, an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets or capsules containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The modulators may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The modulators (e.g., inhibitors) of GPR23 of the invention can be combined or used in combination with other agents useful in the treatment, prevention, suppression or amelioration of metabolic disorders, such as those set forth herein. Such other agents may be administered, by a route and in an amount commonly used therefor, simultaneously or sequentially with a compound of the invention. When a compound of the invention is used contemporaneously with one or more other drugs, a pharmaceutical composition containing such other drugs in addition to the compound of the invention is preferred. Accordingly, the pharmaceutical compositions of the invention include those that also contain one or more other active ingredients or therapeutic agents, in addition to a compound of the invention.

Examples of other therapeutic agents that may be combined with an inhibitor of GPR23, either administered separately or in the same pharmaceutical composition, include, but are not limited to: (a) cholesterol lowering agents such as HMG-CoA reductase inhibitors (e.g., lovastatin, simvastatin, pravastatin, fluvastatin, atorvastatin and other statins), bile acid sequestrants (e.g., cholestyramine and colestipol), vitamin B₃ (also known as nicotinic acid, or niacin), vitamin B₆ (pyridoxine), vitamin B₁₂ (cyanocobalamin), fibric acid derivatives (e.g., gemfibrozil, clofibrate, fenofibrate and benzafibrate), probucol, nitroglycerin, and inhibitors of cholesterol absorption (e.g., beta-sitosterol and acylCoA-cholesterol acyltransferase (ACAT) inhibitors such as melinamide), HMG-CoA synthase inhibitors, squalene epoxidase inhibitors and squalene synthetase inhibitors; (b) antithrombotic agents, such as thrombolytic agents (e.g., streptokinase, alteplase, anistreplase and reteplase), heparin, hirudin and warfarin derivatives, β-blockers (e.g., atenolol), β-adrenergic agonists (e.g., isoproterenol), ACE inhibitors and vasodilators (e.g., sodium nitroprusside, nicardipine hydrochloride, nitroglycerin and enaloprilat); and (c) anti-diabetic agents such as insulin and insulin mimetics, sulfonylureas (e.g., glyburide, meglinatide), biguanides, e.g., metformin (Glucophage®), α-glucosidase inhibitors (acarbose), insulin sensitizers, e.g., thiazolidinone compounds, rosiglitazone (Avandia®), troglitazone (Rezulin®), ciglitazone, pioglitazone (Actos®) and englitazone.

The weight ratio of the inhibitor of GPR23 to the second active agent may be varied and will depend upon the effective dose of each ingredient. Generally, an effective dose of each will be used. Combinations of a compound of the invention and other active ingredients will generally also be within the aforementioned ranges, but in each case, an effective dose of each active ingredient should be used.

C. Therapeutically Effective Doses

The identified modulators (e.g., inhibitors) are administered to a patient at therapeutically effective doses to prevent, treat, or control metabolic disorders mediated, in whole or in part, by a GPR23. The GPR23 modulators are administered to a patient in an amount sufficient to elicit an effective protective or therapeutic response in the patient, i.e., a response that at least partially arrests or slows the symptoms or complications of the disease. An amount adequate to accomplish this is defined as a “therapeutically effective dose.” The dose will be determined by the efficacy of the particular GPR23 modulators employed and the condition of the subject, as well as the body weight or surface area of the area to be treated. The size of the dose also will be determined by the existence, nature, and extent of any adverse effects that accompany the administration of a particular compound or vector in a particular subject.

Toxicity and therapeutic efficacy of the modulators can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index and can be expressed as the ratio, LD₅₀/ED₅₀. Compounds that exhibit large therapeutic indices are preferred. While compounds that exhibit toxic side effects can be used, care should be taken to design a delivery system that targets such compounds to the site of affected tissue to minimize potential damage to normal cells and, thereby, reduce side effects.

The data obtained from cell culture assays and animal studies can be used to formulate a dosage range for use in humans. The dosage of such compounds lies preferably within a range of circulating concentrations that include the ED₅₀ with little or no toxicity. The dosage can vary within this range depending upon the dosage form employed and the route of administration. For any compound used in the methods of the invention, the therapeutically effective dose can be estimated initially from cell culture assays. A dose can be formulated in animal models to achieve a circulating plasma concentration range that includes the IC₅₀ (the concentration of the test compound that achieves a half-maximal inhibition of symptoms) as determined in cell culture. Such information can be used to more accurately determine useful doses in humans. Levels in plasma can be measured, for example, by high performance liquid chromatography (HPLC).

D. Pharmaceutical Compositions

Pharmaceutical compositions for use in the present invention can be formulated by standard techniques using one or more physiologically acceptable carriers or excipients. The compounds and their physiologically acceptable salts and solvates can be formulated for administration by any suitable route, including via inhalation, topically, nasally, orally, parenterally (e.g., intravenously, intraperitoneally, intravesically or intrathecally) or rectally.

For oral administration, the pharmaceutical compositions can take the form of, for example, tablets or capsules prepared by conventional means with pharmaceutically acceptable excipients, including binding agents, for example, pregelatinised maize starch, polyvinylpyrrolidone, or hydroxypropyl methylcellulose; fillers, for example, lactose, microcrystalline cellulose, or calcium hydrogen phosphate; lubricants, for example, magnesium stearate, talc, or silica; disintegrants, for example, potato starch or sodium starch glycolate; or wetting agents, for example, sodium lauryl sulphate. Tablets can be coated by methods well known in the art. Liquid preparations for oral administration can take the form of, for example, solutions, syrups, or suspensions, or they can be presented as a dry product for constitution with water or other suitable vehicle before use. Such liquid preparations can be prepared by conventional means with pharmaceutically acceptable additives, for example, suspending agents, for example, sorbitol syrup, cellulose derivatives, or hydrogenated edible fats; emulsifying agents, for example, lecithin or acacia; non-aqueous vehicles, for example, almond oil, oily esters, ethyl alcohol, or fractionated vegetable oils; and preservatives, for example, methyl or propyl-p-hydroxybenzoates or sorbic acid. The preparations can also contain buffer salts, flavoring, coloring, and/or sweetening agents as appropriate. If desired, preparations for oral administration can be suitably formulated to give controlled release of the active compound.

For administration by inhalation, the compounds may be conveniently delivered in the form of an aerosol spray presentation from pressurized packs or a nebulizer, with the use of a suitable propellant, for example, dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, for example, gelatin for use in an inhaler or insufflator can be formulated containing a powder mix of the compound and a suitable powder base, for example, lactose or starch.

The compounds can be formulated for parenteral administration by injection, for example, by bolus injection or continuous infusion. Formulations for injection can be presented in unit dosage form, for example, in ampoules or in multi-dose containers, with an added preservative. The compositions can take such forms as suspensions, solutions, or emulsions in oily or aqueous vehicles, and can contain formulatory agents, for example, suspending, stabilizing, and/or dispersing agents. Alternatively, the active ingredient can be in powder form for constitution with a suitable vehicle, for example, sterile pyrogen-free water, before use.

The compounds can also be formulated in rectal compositions, for example, suppositories or retention enemas, for example, containing conventional suppository bases, for example, cocoa butter or other glycerides.

Furthermore, the compounds can be formulated as a depot preparation. Such long-acting formulations can be administered by implantation (for example, subcutaneously or intramuscularly) or by intramuscular injection. Thus, for example, the compounds can be formulated with suitable polymeric or hydrophobic materials (for example as an emulsion in an acceptable oil) or ion exchange resins, or as sparingly soluble derivatives, for example, as a sparingly soluble salt.

The compositions can, if desired, be presented in a pack or dispenser device that can contain one or more unit dosage forms containing the active ingredient. The pack can, for example, comprise metal or plastic foil, for example, a blister pack. The pack or dispenser device can be accompanied by instructions for administration.

E. Inhibitors of Gene Expression

GPR23 nucleic acid and polypeptide sequences can be used for diagnosis or prognosis of any of the herein-described metabolic disorders in a patient. For example, the sequence, level, or activity of a GPR23 in a patient can be determined, wherein an alteration, e.g., an increase in the level of expression or activity of the GPR23, or the detection of mutations in the GPR23, e.g., activating mutations, indicates the presence or the likelihood of a metabolic disorder.

In one aspect of the present invention, GPR23 inhibitors can also comprise nucleic acid molecules that inhibit expression of a GPR23. Conventional viral and non-viral based gene transfer methods can be used to introduce nucleic acids encoding engineered GPR23 polypeptides in mammalian cells or target tissues, or alternatively, nucleic acids, e.g., inhibitors of GPR23 activity, such as siRNAs or anti-sense RNAs. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1:13-26 (1994).

In some embodiments, small interfering RNAs are administered. In mammalian cells, introduction of long dsRNA (>30 nt) often initiates a potent antiviral response, exemplified by nonspecific inhibition of protein synthesis and RNA degradation. The phenomenon of RNA interference is described and discussed, e.g., in Bass, Nature 411:428-29 (2001); Elbahir et al., Nature 411:494-98 (2001); and Fire et al., Nature 391:806-11 (1998), where methods of making interfering RNA also are discussed. The siRNAs based upon the GPR23 sequences disclosed herein are less than 100 base pairs, typically 30 bps or shorter, and are made by approaches known in the art. Exemplary siRNAs according to the invention could have up to 29 bps, 25 bps, 22 bps, 21 bps, 20 bps, 15 bps, 10 bps, 5 bps or any integer thereabout or therebetween.

Non-Viral Delivery Methods

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Viral Delivery Methods

The use of RNA or DNA viral-based systems for the delivery of inhibitors of GPR23 to treat metabolic disorders are known in the art. Conventional viral-based systems for the delivery of GPR23 nucleic acid inhibitors can include retroviral, lentivirus, adenoviral, adeno-associated and herpes simplex virus vectors for gene transfer.

In many gene therapy applications, it is desirable that the gene therapy vector be delivered with a high degree of specificity to a particular tissue type, e.g., a joint or the bowel. A viral vector is typically modified to have specificity for a given cell type by expressing a ligand as a fusion protein with a viral coat protein on the virus' outer surface. The ligand is chosen to have affinity for a receptor known to be present on the cell type of interest. For example, Han et al., PNAS 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other pairs of virus expressing a ligand fusion protein and target cell expressing a receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences thought to favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described above. Alternatively, vectors can be delivered to cells ex vivo, such as cells explanted from an individual patient.

Ex vivo cell transfection for diagnostics, research, or for gene therapy (e.g., via re-infusion of the transfected cells into the host organism) is well known to those of skill in the art. In some embodiments, cells are isolated from the subject organism, transfected with GPR23 nucleic acids and re-infused back into the subject organism (e.g., patient). Various cell types suitable for ex vivo transfection are well known to those of skill in the art (see, e.g., Freshney et al., Culture of Animal Cells, A Manual of Basic Technique (3rd ed. 1994)) and the references cited therein for a discussion of how to isolate and culture cells from patients).

Vectors (e.g., retroviruses, adenoviruses, liposomes, etc.) containing therapeutic nucleic acids can also be administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and well known to those of skill in the art, and, although more than one route can be used to administer a particular composition, a particular route can often provide a more immediate and more effective reaction than another route.

### EXAMPLES

The following examples are provided by way of illustration only and not by way of limitation. Those of skill in the art will readily recognize a variety of noncritical parameters that could be changed or modified to yield essentially similar results.

### Example 1

### GPR23 Expression Analysis

Expression levels of human and murine GPR23 were evaluated as follows. Concentrations of human and mouse first strand cDNAs (BD Biosciences; San Jose, Calif.; Human multiple tissue panel I and II, and Mouse multiple tissue panel) were normalized to the mRNA expression levels of four different housekeeping genes (α-tubulin, β-actin, glyceraldehyde-3-phosphate dehydrogenase (GAPDH) and phospholipase A₂). To provide cDNAs not represented in the panels, total RNA was prepared at Amgen Inc. (mouse adipose, mouse hypothalamus,) or purchased from BD BioSciences (human adipose, human pancreas, mouse pancreas) and cDNA was prepared from RNA templates using the Invitrogen SuperScript™ First-Strand Synthesis System (Invitrogen; Carlsbad, Calif.) for RT-PCR. GAPDH and human or mouse GPR23 were amplified in a Perkin Elmer GeneAmp PCR System 2400 (Perkin Elmer; Boston, Mass.) thermocycler using gene-specific primers (human- and mouse-specific GAPDH primers provided by BD Biosciences with the MTP, human GPR23: 5′-ATC TTG GGT CTG ATA ACC AAC AGT G-3′ (SEQ ID NO:5) and human GPR23 3′-TCC CAA TTT GAG ACA GAG TAG CAG-5′ (SEQ ID NO:6); mouse GPR23 5′-CAT GAA AAT GAG AAG TGA GAC GGC-3′ (SEQ ID NO:7) and mouse 3′-GTA TGG TAC AAA GCA TAC CAC AAA C-5′ (SEQ ID NO:8)). PCR products were generated with Advantage II polymerase (BD Biosciences) in a 50 μl reaction containing 5 μl cDNA template. The cycling conditions were 94° C. for 30″; 5 cycles, 94° C.-30″, 72° C.-1.0′; 5 cycles, 94° C.-30″, 70° C.-30″, 72° C.-1.0′; 25 cycles, 95° C.-30″, 68° C.-30″, 72° C.-1.0′. Amplification products were visualized on 1% agarose gels containing ethidium bromide.

GPR23 was found to be expressed in many human tissues, with highest expression in white adipose tissue, ovary and testes (data not shown). In mice, GPR23 was also found to be expressed in numerous tissues, including white adipose tissue, ovary, skeletal muscle, lung, heart and brain, with highest expression in white adipose tissue and ovary (data not shown). Murine brown adipose cDNAs were not included in the panel.

### Example 2

### GPR23 Antisense Studies

GPR23-specific antisense oligos were generated by Isis Pharmaceuticals (Carlsbad, Calif.) and were evaluated for activity in bEND (murine brain endothelial) cells. An antisense oligo (CTCAGTATCATTAGCTTCAA (SEQ ID NO:9)) which markedly reduced expression of GPR23 in cultured cells was used for in vivo studies. An oligo without homology to any known gene was provided as a control (CCTTCCCTGAAGGTTCCTCC (SEQ ID NO:10)). For the studies, male C57 black 6 mice were fed a high fat diet for 12 weeks (from 3 weeks of age) and analyzed for parameters of diet induced obesity. Animals that show appropriate symptoms of diet induced obesity (e.g., hyperinsulemia and weight gain with no signs of illness) were divided into 5 groups (10 animals/group) and treated as described below:

Group 1 (saline treated control): saline IP injection weekly for 7 weeks;

Group 2 (universal oligo control): oligo IP injection 50 mg/kg body weight, weekly for 7 weeks;

Group 3 (GPR23 antisense oligo (ASO) treated): oligo IP injection, 25 mg/kg body weight, weekly for 7 weeks;

Group 4 (GPR23 ASO treated): oligo IP injection, 50 mg/kg body weight, weekly for 7 weeks; and

Group 5: (rosiglitizone treated control): 50 mg/kg body weight for 7 weeks.

Fed glucose measurements were made at the beginning, midpoint and endpoint of the study. A fasting GTT measurement was done at the midpoint and endpoint of the study. Fed plasma samples were tested for insulin, total cholesterol, free fatty acids, triglycerides, and liver enzymes. At the end of the study, animals were sacrificed and tissue samples (liver, white adipose tissue, brown adipose tissue) were taken for histological evaluation and GPR23 mRNA quantitation.

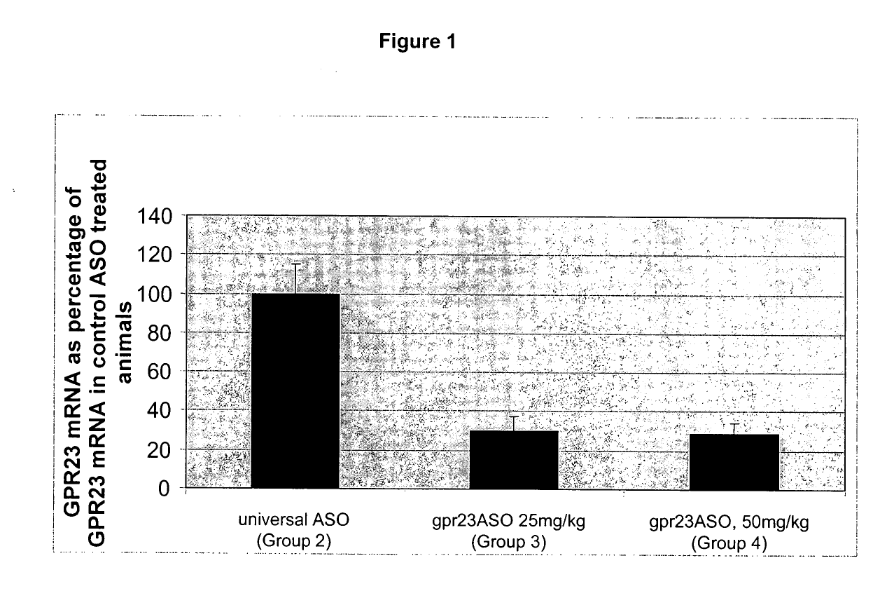

FIG. 1 depicts data, generated using real time PCR analysis, comparing GPR23 mRNA levels in various antisense oligo treated groups. As shown in FIG. 1, antisense oligo treated animals (Group 3 and Group 4) reduced GPR23 mRNA levels ˜70% relative to control oligo treated animals (Group 2) in white adipose tissue.

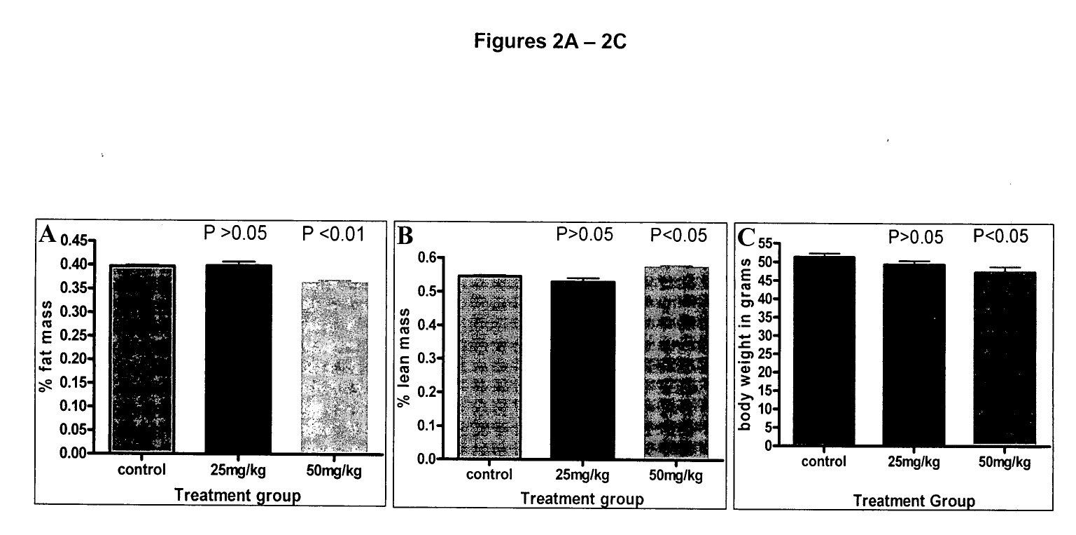

As shown in FIGS. 2A-C, GPR23 antisense oligo treated animals at the 50 mg/kg dose (Group 4) showed a statistically significant decrease in fat mass (FIG. 2A) and a statistically significant increase in lean mass (FIG. 2B) when compared to animals treated with the control oligo (Group 2). Group 4 animals also weighed slightly less than control animals (FIG. 2C).

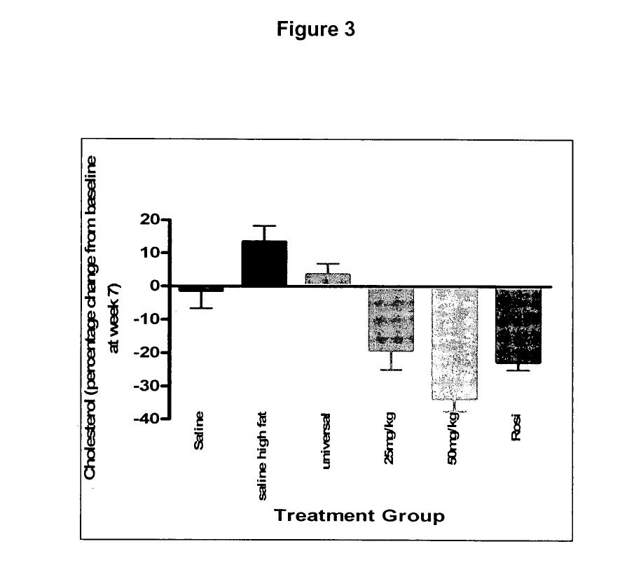

FIG. 3 depicts serum cholesterol levels, as a percentage of change from baseline levels at week 7, in each of the groups. GPR23 antisense oligo treated animals had fed glucose levels similar to controls, and glucose tolerance tests were not affected by antisense oligo treatment. Serum cholesterol levels in treated animals were lowered relative to controls at both the 25 mg/kg and 50 mg/kg doses (Groups 3 and 4, respectively). Though not statistically significant, triglyceride and free fatty acid levels were also lower in both Group 3 and Group 4 treatment groups (data not shown). Furthermore, liver fat scores were lower in both Group 3 and Group 4 treatment groups, whereas liver enzymes (a parameter of toxicity) were not significantly different in the Group 3 and Group 4 treatment groups compared to values determined for control groups (Groups 1 and 2).

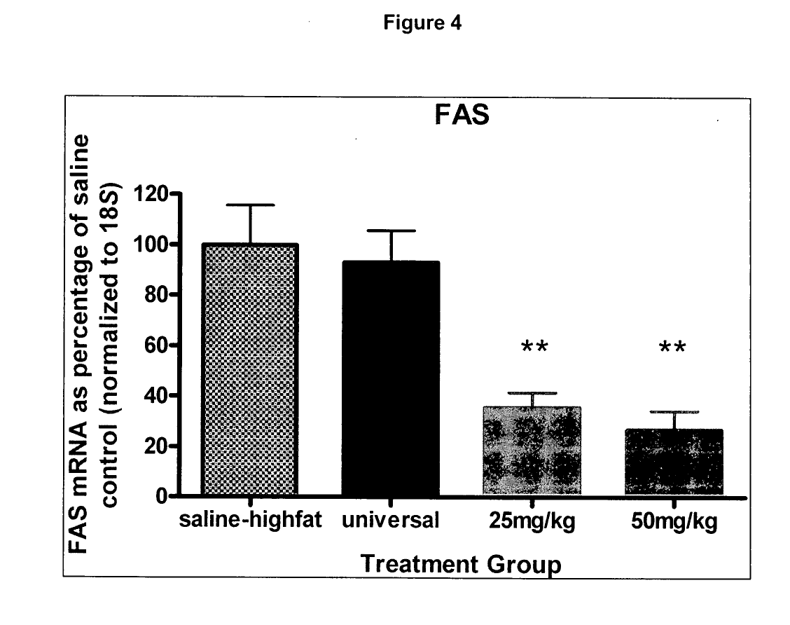

Gene profiling experiments were performed to provide additional insight into the mechanism of action associated with GPR23. These experiments, conducted using real time PCR analysis of adipose tissue, indicated that GPR23 knockdown may affect mRNA levels of gene products in pathways of lipid synthesis, but not in pathways of lipolysis or adipocyte differentiation. In particular, as set forth in FIG. 4, fatty acid synthase mRNA levels were significantly reduced in a dose-dependent manner in white adipose tissue in both Group 3 and Group 4 treatment groups. In contrast, levels of hormone sensitive lipase and PPAR-gamma did not change significantly.

### Example 3

### cAMP Assay

Cell lines expressing human GPR23 were generated as follows. Human GPR23 coding sequence was amplified by PCR from genomic DNA using the Expand Long Template DNA Polymerase Kit (Roche Applied Science, USA) and the following primers: 5′-GGG GTA CCA CCA TGG ATT ACA AGG ATG ACG ACG ATA AGG GTG ACA GAA GAT TCA TTG-3′ (SEQ ID NO:11) and 3′-ATA AGA ATG CGG CCG CCT AAA AGG TGG ATT CTA GC-5′ (SEQ ID NO:12). The PCR product was digested with Kpn1 and Not1 and ligated into pcDNA 3.1+Hygro (Invitrogen, Carlsbad, Calif.), generating a human GPR23 clone with a 5′-flag tag. Human GPR23 sequence was verified by sequencing. An untagged version of this clone was generated by PCR from the tagged clone, using the following oligonucleotides: 5′-ACT TAA GCT TGG TAC CAC CAT GGG TGA CAG AAG ATT C (SEQ ID NO:13) and 3′-CCT CTA GAC TCG AGC GGC CGC TAA AAG GTG GAT TCT AG (SEQ ID NO:14). The PCR product was digested with Kpn1 and Not1 and ligated into pcDNA 3.1+Hygro.

Cell lines in which expression of human GPR23 was inducible were generated using the T-Rex™ system (Invitrogen). The human GPR23 coding sequence was excised from the pcDNA clone by restriction with Kpn1 and Not1 and was ligated into the expression vector pcDNA4/TO (Invitrogen). Human GPR23 sequence in the construct was verified by sequencing and the DNA was transfected into T-Rex™-CHO cells (Invitrogen) using Lipofectamine 2000 (Invitrogen) and a standard protocol. A pool of cells stably expressing human GPR23 was selected in growth media (F12+10% FBS (Tet free, HyClone; Logan, Utah), 1× Glutamine (Invitrogen), 100 ug/ml Blasticidin (Invitrogen) containing 250 ug/ml zeocin. Clonal cell lines were isolated by dilution. After isolation and expansion of clones, GPR 23 expression was verified and quantitated by bDNA technology. Two clonal cell lines were chosen for further analysis.

T-Rex HuGPR23 μl cells were cultured in F12 media supplemented with 10% FBS, 100 IU/ml penicillin, 100 ug/ml streptomycin, 1 mM glutamate, 10 ug/ml blastocidin and 250 ug/ml zeocin. For the cAMP assay, cells were serum starved in growth media without FBS for 24 hours prior to performing the assay. Tetracycline or doxycycline (1 ug/ml) and Pertussis toxin (10 ng/ml) were also added to the media. Cells were harvested with Versene (Invitrogen), washed 1× with stimulation buffer (1×HBSS, 5 mM HEPES, 0.1% BSA, 0.2 mM IBMX) and resuspended in stimulation buffer at a concentration of 2×10⁶ cells/ml. Next, 12 ul of the cell suspension containing Alex Fluor 647-labeled antibody (PerkinElmer; Wellesley, Mass.) were applied to 96-well white assay plates (Costar #3693, 96 well ½ area; Fischer Scientific, USA) and incubated at 37° C. while ligand was prepared. Reaction was initiated by the addition of 12 ul of 2× ligand solution. After 30 minutes of incubation at 37° C., cAMP levels were determined using the LANCE cAMP 384 kit (PerkinElmer) and a TRF detection instrument (Packard Discovery HTRF).

The examples set forth above are provided by way of illustration only and not by way of limitation. Those of skill in the art will readily recognize a variety of non-critical parameters that could be changed or modified to yield essentially the same results.

All publications, patents, and patent applications cited in this specification are herein incorporated by reference as if each individual publication, patent or patent application were specifically and individually indicated to be incorporated by reference.

### SEQUENCES

```
SEQ ID NO:1 Human GPR23 Nucleic Acid Sequence
      1 cctaccggtc catagtgtca gagtggtgaa cccctgcagc cagcaggcct cctgaaaaaa
    61 aagtccatgg gtgacagaag attcattgac ttccaattcc aagattcaaa ttcaagcctc
  121 agacccaggt tgggcaatgc tactgccaat aatacttgca rtgttgatga ttccttcaag
  181 tataatctca atggtgctgt ctacagtgtt gtattcatct tgggtctgat aaccaacagt
  241 gtctctctgt ttgtcttctg tttccgcatg aaaatgagaa gtgagactgc tatttttatc
  301 accaatctag ctgtctctga tttgcttttt gtctgtacac taccttttaa aatattttac
  361 aacttcaacc gccactggcc ttttggtgac accctctgca agatctctgg aactgcattc
  421 cttaccaaca tctatgggag catgctcttt ctcacctgta ttagtgtgga tcgtttcctg
  481 gccattgtct atccttttcg atctcgtact attaggacta ggaggaattc tgccattgtg
  541 tgtgctggtg tctggatcct agtcctcagt ggcggtattt cagcctcttt gttttccacc
  601 actaatgtca acaatgcaac caccacctgc tttgaaggct tctccaaacg tgtctggaag
  661 acttatttat ccaagatcac aatatttatt gaagttgttg ggtttatcat tcctctaata
  721 ttgaatgtct cttgctcttc tgtggtgctg agaactcttc gcaagcctgc tactctgtct
  781 caaattggga ccaataagaa aaaagtactg aaaatgatca cagtacatat ggcagtcttt
  841 gtggtatgct ttgtacccta caactctgtc ctcttcttgt atgccctggt gcgctcccaa
  901 gctattacta attgcttttt ggaaagattt gcaaagatca tgtacccaat caccttgtgc
  961 cttgcaactc tgaactgttg ttttgaccct ttcatctatt acttcaccct tgaatccttt
1021 cagaagtcct tctacatcaa tgcccacatc agaatggagt ccctgtttaa gactgaaaca
1081 cctttgacca caaagccttc ccttccagct attcaagagg aagtgagtga tcaaacaaca
1141 aataatggtg gtgaattaat gctagaatcc accttttagg tatgagaaat gtgttcaggt
1201 ccagatatgg tttctcctat aatttttcct atgctataaa ctaaagattt gaagctaatg
1261 atactgagaa taatgcacca aatccagtca gatacatttg tttgaaggta tactgtagag
1321 tttttattgc tgttttgttc agtaattata ggtcaaatct aattacaaca accaagatgg
1381 attgccaaac tcttctgctt ggttggaatt tcattgtatc gcattatcca ggtggctagt
1441 ggcatttgat aatatagaga tgactttgaa actttcaaaa aggtatttct attccaatga
1501 tatttggtaa ttaggttggg cctataaata tagaacaaat tcagggattt ttaaaaaatt
1561 gtgttactac tgatatatgc tagttttatt ttattttttt ggactgtcat tgagtttatt
1621 ttagcacaag aatattttta gcctaacatt attaataaga aatgtgtcaa atttttaaca
1681 ttggtaaaat atgttatgtg cattttgaaa acagaaaaca aattgcgttg gcatgtacgt
1741 gggtgggaag aaaaagaaaa ttaacaggat ttacacaatt ataatcacca gcagtgtgag
1801 tttaaaaaac ttcgttgttt ttacaccaaa ttaaaatttt catgtcaaac ttcaaagcca
1861 gaaagctgct aaatacgtgt ctggcaggta aaagctggaa aattacttaa aacaggaaag
1921 tgtcaataaa aaaacttgag caacaccaac atattttttc ttaaaatgtc acgttatctt
1981 cattttggga aactaggttc tataaaatat ttatcctccc tgttatactt tggagcacag
2041 cacagecaga aaggggctgc atttgtgccc aggtcaggag caaattgaaa aaaaaaataa
2101 agtaatacta aaaaatcaaa ctataaaccc aaaacattta ttaaaacctg aattaatcct
2161 ttttggaggg aggagtagag atatataacc tgaaaatact tattctttct tatcgaattt
2221 tggagcctaa tatagccagg agctgctgaa tttgtgcccc tggattggaa ccaaataaaa
2281 aaaaaaaaaa aaaattcct
SEQ ID NO:2 Human GPR23 Protein Sequence
MGDRRFIDFQFQDSNSSLRPRLGNATANNTCIVDDSFKYNLNGVYSVVFILGLITNSVSLFVFCFRMKMR
SETAIFITNLAVSDLLFVCTLPFKIFYNFNRHWPFGDTLCKISGTAFLTNIYGSMLFLTCISVDRFLAIV
YPFRSRTIRTRRNSAIVCAGVWILVLSGGISASLFSTTNVNNATTTCFEGFSKRVWKTYLSKITIFIEVV
GFIIPLILNVSCSSVVLRTLRKPATLSQIGTNKKKVLKMITVHMAVFVVCFVPYNSVLFLYALVRSQAIT
NCFLERFAKIMYPITLCLATLNCCFDPFIYYFTLESFQKSFYINAHIRMESLFKTETPLTTKPSLPAIQE
EVSDQTTNNGGELMLESTF
SEQ ID NO:3 mouse GPR23 nucleic acid sequence
      1 tagactttgg gccttttctt gtgtcctgtt tgttaaaggc atgcgggctc cagcattaaa
    61 gagggctagt ccttaacaaa gggaaagcga taaatgtaaa taagctcaca ttttcagaat
  121 gagcggtttg cagtaaggag ctgcggcagc ccagagtctg ctctttttgg gctgggctaa
  181 cctttccctg ttttttgttt tttgttttgt tttgtttttg ttttttatgg ataaaaatat
  241 gcgcttccga agtgcgagtt gccagtttac acgtttatta gctaactatc tacaggcatg
  301 agcacattct ctcatctagc acactctttc ttgggcactc aattgaggaa ctctctgatc
  361 gtctgcctcc agaaaattca ttgattatcc aagtctcaga taaatctggt gccagagttt
  421 ggtttgaact aactaatgaa gaaagcattc tctactggtc ctcagtctca agagtggtga
  481 acccctgcac ctagcaggct ctctgggaaa aaaaaatcca tgggtgacag aagatttatt
  541 gacttccaat tccaagattt aaattcaagt ctcagaccca ggttgggaaa tgcaactgcc
  601 aataatactt gcattgttga tgattccttc aagtataatt tgaatggtgc tgtctatagt
  661 gttgtattca tcctgggtct aataaccagc agtgcctccc tgtttgtctt ctgcttccgc
  721 atgaaaatga gaagtgagac ggctattttc atcaccaacc tggccctctc tgatttgctt
  781 tttgtttgta ccctaccttt caaaatattt tacaacttta atcgccactg gccttttggt
  841 gacaccctct gtaagatctc agggactgcg ttcctcacca acatctatgg gagcatgctc
  901 ttcctcacct gcatcagtgt ggatcgtttc ctagccattg tctatccctt ccgatcgcgt
  961 accatcagga ccaggaggaa ttccgccatt gtgtgcgctg gagtctggat cctagtcctc
1021 agtggtggta tttcagcttc tttgttctcc accactaatg tcaacaatgc gaccaccact
1081 tgctttgaag gcttctccaa acgtgtctgg aagacatacc tgtccaagat cactatattc
1141 attgaagttg ttggattcat cattcctctg atattgaatg tttcttgttc ttctgtggtg
1201 cttagaaccc tccgcaagcc tgcaacattg tctcagattg ggaccaataa aaaaaaagtg
1261 ttgaagatga tcacagtgca tatggcagtg tttgtggtat gctttgtacc atacaactcc
1321 gttctctttt tatatgcctt ggtacgctcc caagccatta ctaattgctt attggaaagg
1381 tttgcaaaga tcatgtaccc aattaccttg tgccttgcaa ctctgaattg ttgctttgat
1441 ccttttatct attacttcac tcttgaatcc tttcagaagt ccttttatat caatacacat
1501 ataaggatgg agtcgctgtt taagactgag acacctctga cccccaaacc ttcccttcca
1561 gctatccaag aggaagttag tgatcaaaca acaaataatg gtggtgaatt aatgctggaa
1621 tccaccttct aggtaccaga attgtctttc aggttcagct acagtgtctc ttatgatttt
1681 tttcctatgc tataaatagg agaaacaaat tgaagctaat gatactgaga atagagtaat
1741 gtaccaaatg cagtcagata catttgtttg aacactattg tacatattct gttttgttca
1801 gtaattatag gtcaagtcta attacaacaa ccaaaacaga tcagcctctt ctgttgagtt
1861 gacttttcat tacctaaatg accagtggtc ttgactttta gtgatgtgag ggttattttt
1921 aaacttaaaa aaaaaggcat tccagtaatt ttggtaattg ggttgggcct ataaatatag
1981 aacaaattca gggattattt aaaaacatct gtgttactac tgatatatgc tagtattttt
2041 ttcctttttt gaattaatat tgaatttatt ttaaaaaaag aactattttt acctaatctt
2101 aataagacat actgagaaag agaaatgtgt tgaattttaa aatattggca aattttacct
2161 agattttaaa aacctaaatg aagtgtttga atgaatatgg gtgggaaatt tggaatttag
2221 acaacattta cgcatttata ataaccacaa ttagtgtcag cttttaaaac tttcttttta
2281 aaataattct agaattttca tatgaaattg ttaatcctga aaggtgctac ttatgtgcct
2341 ggcaggtata aaatggaaaa ctcataaaat taacagtgtc aatttaaaaa aaaaaaaact
2401 tcaagcaaca ctatattatt tcttaagatt ttcatttatc ctttatgggg gtggggattg
2461 gcttgtagaa aatatttatt cttcatgtta aatgttgggg acacattaca gccagagagc
2521 tacagtattt gtgcccaggt caggagtaaa ttgaaaaagt aagtgaatag aatagtagca
2581 gcaagatatc ttaaagctta tattagtagt ttttaaggtg gtggttagat agctgtaatt
2641 ttgaaatcca tactctcttc tgtacatttt ggagcacatt ttacccaagg ccgctgctga
2701 atttgtgctc aggtcgggag catattgaaa aagatgtgta c
SEQ ID NO:4 mouse GPR23 polypeptide sequence
MGDRRFIDFQFQDLNSSLRPRLGNATANNTCIVDDSFKYNLNGAVYSVVFILGLITSSASLFVFCFRMKM
RSETAIFITNLALSDLLFVCTLPFKIFYNFNRHWPFGDTLCKISGTAFLTNIYGSMLFLTCISVDRFLAI
VYPFRSRTIRTRRNSAIVCAGVWILVLSGGISASLFSTTNVNNATTTCFEGFSKRVWKTYLSKITIFIEV
VGFIIPLILNVSCSSVVLRTLRKPATLSQIGTNKKKVLKMITVHMAVFVVCFVPYNSVLFLYALVRSQAI
TNCLLERFAKIMYPITLCLATLNCCFDPFIYYFTLESFQKSFYINTHIRMESLFKTETPLTPKPSLPAIQ
EEVSDQTTNNGGELMLESTF
SEQ ID NO:5 human GPR23:
5′-ATC TTG GGT CTG ATA ACC AAC AGT G-3′
SEQ ID NO:6 human GPR23
3′-TCC CAA TTT GAG ACA GAG TAG CAG-5′
SEQ ID NO:7 mouse GPR23
5′-CAT GAA AAT GAG AAG TGA GAC GGC-3′
SEQ ID NO:8 mouse GPR23
3′-GTA TGG TAC AAA GCA TAC CAC AAA C-5′
SEQ ID NO:9
CTCAGTATCATTAGCTTCAA
SEQ ID NO:10
CCTTCCCTGAAGGTTCCTCC
SEQ ID NO:11
5′-GGG GTA CCA CCA TGG ATT ACA AGG ATG ACG ACG ATA AGG GTG ACA GAA GAT
TCA TTG-3′
SEQ ID NO:12
3′-ATA AGA ATG CGG CCG CCT AAA AGG TGG ATT CTA GC-5′
SEQ ID NO:13
5′-ACT TAA GCT TGG TAC CAC CAT GGG TGA CAG AAG ATT C
SEQ ID NO:14
3′-CCT CTA GAC TCG AGC GGC CGC TAA AAG GTG GAT TCT AG
```

What is claimed is:

1. A method of identifying a compound to treat or prevent a metabolic disorder, the method comprising:
a) contacting at least one candidate compound with a polypeptide, wherein the polypeptide:
(i) comprises at least 200 contiguous amino acids of SEQ ID NO:2 or SEQ ID NO:4,
(ii) has at least 90% identity to a polypeptide of SEQ ID NO:2 or SEQ ID NO:4,
(iii) comprises SEQ ID NO 2 or SEQ ID NO:4,
(iv) consists of SEQ ID NO:2 or SEQ ID NO:4; or
(v) comprises GPR23;
b) determining the functional effect of the candidate compound on the activity of the polypeptide; and
c) selecting a compound from the at least one candidate compound that inhibits the polypeptide.

2. The method of claim 1, further comprising the steps of:
d) administering a compound identified in step c) to an animal;
e) determining the effect of the compound on the onset or symptoms of a metabolic disorder; and
f) selecting a compound that delays the onset or reduces the severity of the symptoms of the metabolic disorder.

3. The method of claim 1, wherein the step of determining the functional effect comprises measuring a change in intracellular calcium or cAMP.

4. The method of claim 1, wherein the step of determining the functional effect comprises performing a binding assay.

5. The method of claim 1, wherein the candidate compound is an antibody.

6. The method of claim 1, wherein the candidate compound is a small molecule.

7. The method of claim 1, wherein the polypeptide comprises SEQ ID NO:2.

8. The method of claim 1, wherein the polypeptide comprises SEQ ID NO:4.

9. The method of claim 1, wherein the polypeptide consists of SEQ ID NO:2.

10. The method of claim 1, wherein the polypeptide consists of SEQ ID NO:4.

11. The method of claim 1, wherein the polypeptide comprises GPR23.

12. The method of claim 1, wherein the metabolic disorder is selected from the group consisting of dyslipidemia, metabolic syndrome, obesity and obesity related disorders.

13. A method of identifying a compound to treat or prevent dyslipidemia, metabolic syndrome, obesity or an obesity-related disorder, comprising:
a) contacting at least one candidate compound with a polypeptide, wherein the polypeptide:
(i) comprises at least 200 contiguous amino acids of SEQ ID NO:2 or SEQ ID NO:4,
(ii) has at least 90% identity to a polypeptide of SEQ ID NO:2 or SEQ ID NO:4,
(iii) comprises SEQ ID NO 2 or SEQ ID NO:4,
(iv) consists of SEQ ID NO:2 or SEQ ID NO:4; or
(v) comprises GPR23;
b) determining the functional effect of the candidate compound on the activity of the polypeptide; and
c) selecting a compound from the at least one candidate compound that inhibits the polypeptide.

14. The method of claim 13, further comprising the steps of
d) administering a compound identified in step c) to an animal;
e) determining the effect of the compound on the onset or symptoms of dyslipidemia, metabolic syndrome or obesity; and
f) selecting a compound that delays the onset or reduces the severity of the symptoms of dyslipidemia, metabolic syndrome or obesity.

15. The method of claim 13, wherein the step of determining the functional effect comprises measuring a change in intracellular calcium or cAMP.

16. The method of claim 13, wherein the step of determining the functional effect comprises performing a binding assay.

17. The method of claim 13, wherein the candidate compound is an antibody.

18. The method of claim 13, wherein the candidate compound is a small molecule.

19. The method of claim 13, wherein the polypeptide comprises SEQ ID NO:2.

20. The method of claim 13, wherein the polypeptide comprises SEQ ID NO:4.

21. The method of claim 13, wherein the polypeptide consists of SEQ ID NO:2.

22. The method of claim 13, wherein the polypeptide consists of SEQ ID NO:4.

23. The method of claim 13, wherein the polypeptide comprises GPR23.

24. A method of treating a metabolic disorder, the method comprising administering a compound identified by the method of claim 1 or claim 13.

25. The method of claim 24, wherein the compound is an antibody.

26. The method of claim 24, wherein the compound is a small molecule.

27. The method of claim 24, wherein the metabolic disorder is selected from the group consisting of dyslipidemia, metabolic syndrome, obesity and obesity-related disorders.

28. A method of treating a metabolic disorder, the method comprising administering a therapeutically effective amount of a compound that modulates GPR23.

29. The method of claim 28, wherein the compound is an antibody.

30. The method of claim 28, wherein the compound is a small molecule.

31. The method of claim 28, wherein the compound is an inhibitor.

32. The method of claim 28, wherein the metabolic disorder is selected from the group consisting of dyslipidemia, metabolic syndrome, obesity and obesity-related disorders.
